SEQUENCE LISTING (37 sequences)

SEQ ID NO: 1 - DNA, 899 bp, Human alphaherpesvirus 2
  source 1..899: mol_type = genomic DNA
cgtcaaggcggggtggtacgccagcacgagccacgaggcgcggctgctgagacgcctgaaccaccccgcgatcctacccctcctggacctgcacgtcgtttctggggtcacgtgtctggtcctccccaagtatcactgcgacctgtatacctatctgagcaagcgcccgtctccgttgggccacctacagataaccgcggtctcccggcagctcttgagcgccatcgactacgtccactgcaaaggcatcatccaccgcgatattaagaccgagaacatcttcatcaacacccccgagaacatctgtctgggggactttggggcggcgtgctttgtgcgcgggtgtcgatcgagccccttccattacgggatcgcaggcaccatcgatacaaacgcccccgaggtcctggccggggatccgtacacccaggtaatcgacatctggagcgccggcctggtgatctttgagaccgccgtccacaccgcgtccttgttctcggccccgcgcgaccccgaaaggcggccgtgcgacaaccagatcgcgcgcatcatccgacaggcccaggtacacgtcgacgagtttccgacgcacgcggaatcgcgcctcaccgcgcactaccgctcgcgggcggccgggaacaatcgtccggcgtggacccgaccggcgtggacccgctactacaagatccacacagacgtcgaatatctcatatgcaaagcccttacctttgacgcggcgctccgcccaagcgccgcggagttgctgcgcctgccgctatttcaccctaagtgaccccgctccccccggggggcgtggaggggggggctggttggatgtttttgcacaaaaagacgcggccctcgggctttggtgtttttggcaccttgccgcccggcgt

SEQ ID NO: 2 - DNA, 100 bp, Human alphaherpesvirus 1
  source 1..100: mol_type = genomic DNA
caaatatttttattgcaactccctgttttaggtacaataaaaacaaaacatttcaaacaaatcgcccctcgtgttgtccttctttgctcatggccggcgg

SEQ ID NO: 3 - DNA, 627 bp, Synthetic construct
  source 1..627: mol_type = other DNA
taatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtatataagcagagctctccctatcagtgatagagatctccctatcagtgatagagatcgtcgacgagctcgcgtgtcgcatttgcacctcggcactcggagcgagacgcagcagccaggcagactcgggccgccccctctccgcatcaccacagaagccccgcctacgttgcgacccccaggg

SEQ ID NO: 4 - DNA, 900 bp, Human alphaherpesvirus 2
  source 1..900: mol_type = genomic DNA
ggggtgggggtggggggcgagaaacgatgaaggacgggaaagggaacagcgaccaaatgtcacgataagaacaataaacctgtgacgtcaatcagatatgtgagtttggttgtgttttgtgggactgggggcggggggtgggaggtatcagtgggtgacagagtcttttaaaagacgtgtcccggggccctcgagatgcgcaacttttggccacacagagaaaggcccccagacgaagtcacccgggtccccgaacaaaaacaaaaaccttgaccgccgccggggggcgtgcctgttgttttggtctcaatggatcggtatgccgttcggacctgggggattgtgggaatcctcgggtgtgctgctgttggggccgcacccaccggccccgcgtccgatacaacaaacgcgaccgcacgcctccccacgcaccccccactcatccgttccgggggctttgccgtccccctcatcgtgggggggctgtgtctcatgattctggggatggcgtgtctactcgaggtcctgcgtcgcctgggtcgcgagttggcgaggtgctgcccccacgcgggccaatttgccccatgatttttcgcctttctggccttgcccccaccccatcgccccgattgtgtgtcgggtgcccggggtacagcagctatggagcggtcggtaatataactttggttgtcgccacacgccccgtgccgggcatgggttgtgcgggaaggacgaaataatccggcgatccccaagcgtaccaactgggggggggggggggggggaaaagaaactaaaaacacatcaagcccacaacccatcccacaatgggggttatggcggacccaccgcaccaccatactccgattcgaccacatatgcaaccaaatcacccccaga

SEQ ID NO: 5 - DNA, 67 bp, Synthetic construct
  source 1..67: mol_type = other DNA
aagcttggtcgatatctatcggagctgtgctagcgccactggcggccgctgcagcgagcggaattct

SEQ ID NO: 6 - DNA, 2647 bp, Synthetic construct
  source 1..2647: mol_type = other DNA
cagctgcgtcaaggcggggtggtacgccagcacgagccacgaggcgcggctgctgagacgcctgaaccaccccgcgatcctacccctcctggacctgcacgtcgtttctggggtcacgtgtctggtcctccccaagtatcactgcgacctgtatacctatctgagcaagcgcccgtctccgttgggccacctacagataaccgcggtctcccggcagctcttgagcgccatcgactacgtccactgcaaaggcatcatccaccgcgatattaagaccgagaacatcttcatcaacacccccgagaacatctgtctgggggactttggggcggcgtgctttgtgcgcgggtgtcgatcgagccccttccattacgggatcgcaggcaccatcgatacaaacgcccccgaggtcctggccggggatccgtacacccaggtaatcgacatctggagcgccggcctggtgatctttgagaccgccgtccacaccgcgtccttgttctcggccccgcgcgaccccgaaaggcggccgtgcgacaaccagatcgcgcgcatcatccgacaggcccaggtacacgtcgacgagtttccgacgcacgcggaatcgcgcctcaccgcgcactaccgctcgcgggcggccgggaacaatcgtccggcgtggacccgaccggcgtggacccgctactacaagatccacacagacgtcgaatatctcatatgcaaagcccttacctttgacgcggcgctccgcccaagcgccgcggagttgctgcgcctgccgctatttcaccctaagtgaccccgctccccccggggggcgtggaggggggggctggttggatgtttttgcacaaaaagacgcggccctcgggctttggtgtttttggcaccttgccgcccggcgtaccggtactagtcaattgcaaatatttttattgcaactccctgttttaggtacaataaaaacaaaacatttcaaacaaatcgcccctcgtgttgtccttctttgctcatggccggcggggtaccgttaacatacgtaacgcgtaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtatataagcagagctctccctatcagtgatagagatctccctatcagtgatagagatcgtcgacgagctcgcgtgtcgcatttgcacctcggcactcggagcgagacgcagcagccaggcagactcgggccgccccctctccgcatcaccacagaagccccgcctacgttgcgacccccagggaagcttggtcgatatctatcggagctgtgctagcgccactggcggccgctgcagcgagcggaattctggggtgggggtggggggcgagaaacgatgaaggacgggaaagggaacagcgaccaaatgtcacgataagaacaataaacctgtgacgtcaatcagatatgtgagtttggttgtgttttgtgggactgggggcggggggtgggaggtatcagtgggtgacagagtcttttaaaagacgtgtcccggggccctcgagatgcgcaacttttggccacacagagaaaggcccccagacgaagtcacccgggtccccgaacaaaaacaaaaaccttgaccgccgccggggggcgtgcctgttgttttggtctcaatggatcggtatgccgttcggacctgggggattgtgggaatcctcgggtgtgctgctgttggggccgcacccaccggccccgcgtccgatacaacaaacgcgaccgcacgcctccccacgcaccccccactcatccgttccgggggctttgccgtccccctcatcgtgggggggctgtgtctcatgattctggggatggcgtgtctactcgaggtcctgcgtcgcctgggtcgcgagttggcgaggtgctgcccccacgcgggccaatttgccccatgatttttcgcctttctggccttgcccccaccccatcgccccgattgtgtgtcgggtgcccggggtacagcagctatggagcggtcggtaatataactttggttgtcgccacacgccccgtgccgggcatgggttgtgcgggaaggacgaaataatccggcgatccccaagcgtaccaactgggggggggggggggggggaaaagaaactaaaaacacatcaagcccacaacccatcccacaatgggggttatggcggacccaccgcaccaccatactccgattcgaccacatatgcaaccaaatcacccccagatctaga

SEQ ID NO: 7 - DNA, 2750 bp, Synthetic construct
  source 1..2750: mol_type = other DNA
acttaagcttgccaccatgagaggcggcggcctgatctgcgccctggtggtgggcgccctggtggccgccgtggccagcgccgcccccgccgcccccgccgcccccagagccagcggcggcgtggccgccaccgtggccgccaacggcggccccgccagcagacccccccccgtgcccagccccgccaccaccaaggccagaaagagaaagaccaagaagccccccaagagacccgaggccacccccccccccgacgccaacgccaccgtggccgccggccacgccaccctgagagcccacctgagagagatcaaggtggagaacgccgacgcccagttctacgtgtgccccccccccaccggcgccaccgtggtgcagttcgagcagcccagaagatgccccaccagacccgagggccagaactacaccgagggcatcgccgtggtgttcaaggagaacatcgccccctacaagttcaaggccaccatgtactacaaggacgtgaccgtgagccaggtgtggttcggccacagatacagccagttcatgggcatcttcgaggacagagcccccgtgcccttcgaggaggtgatcgacaagatcaacgccaagggcgtgtgcagaagcaccgccaagtacgtgagaaacaacatggagaccaccgccttccacagagacgaccacgagaccgacatggagctgaagcccgccaaggtggccaccagaaccagcagaggctggcacaccaccgacctgaagtacaaccccagcagagtggaggccttccacagatacggcaccaccgtgaactgcatcgtggaggaggtggacgccagaagcgtgtacccctacgacgagttcgtgctggccaccggcgacttcgtgtacatgagccccttctacggctacagagagggcagccacaccgagcacaccagctacgccgccgacagattcaagcaggtggacggcttctacgccagagacctgaccaccaaggccagagccaccagccccaccaccagaaacctgctgaccacccccaagttcaccgtggcctgggactgggtgcccaagagacccgccgtgtgcaccatgaccaagtggcaggaggtggacgagatgctgagagccgagtacggcggcagcttcagattcagcagcgacgccatcagcaccaccttcaccaccaacctgacccagtacagcctgagcagagtggacctgggcgactgcatcggcagagacgccagagaggccatcgacagaatgttcgccagaaagtacaacgccacccacatcaaggtgggccagccccagtactacctggccaccggcggcttcctgatcgcctaccagcccctgctgagcaacaccctggccgagctgtacgtgagagagtacatgagagagcaggacagaaagcccagaaacgccacccccgcccccctgagagaggcccccagcgccaacgccagcgtggagagaatcaagaccaccagcagcatcgagttcgccagactgcagttcacctacaaccacatccagagacacgtgaacgacatgctgggcagaatcgccgtggcctggtgcgagctgcagaaccacgagctgaccctgtggaacgaggccagaaagctgaaccccaacgccatcgccagcgccaccgtgggcagaagagtgagcgccagaatgctgggcgacgtgatggccgtgagcacctgcgtgcccgtggcccccgacaacgtgatcgtgcagaacagcatgagagtgagcagcagacccggcacctgctacagcagacccctggtgagcttcagatacgaggaccagggccccctgatcgagggccagctgggcgagaacaacgagctgagactgaccagagacgccctggagccctgcaccgtgggccacagaagatacttcatcttcggcggcggctacgtgtacttcgaggagtacgcctacagccaccagctgagcagagccgacgtgaccaccgtgagcaccttcatcgacctgaacatcaccatgctggaggaccacgagttcgtgcccctggaggtgtacaccagacacgagatcaaggacagcggcctgctggactacaccgaggtgcagagaagaaaccagctgcacgacctgagattcgccgacatcgacaccgtgatcagagccgacgccaacgccgccatgttcgccggcctgtgcgccttcttcgagggcatgggcgacctgggcagagccgtgggcaaggtggtgatgggcgtggtgggcggcgtggtgagcgccgtgagcggcgtgagcagcttcatgagcaaccccttcggcgccctggccgtgggcctgctggtgctggccggcctggtggccgccttcttcgccttcagatacgtgctgcagctgcagagaaaccccatgaaggccctgtaccccctgaccaccaaggagctgaagaccagcgaccccggcggcgtgggcggcgagggcgaggagggcgccgagggcggcggcttcgacgaggccaagctggccgaggccagagagatgatcagatacatggccctggtgagcgccatggagagaaccgagcacaaggccagaaagaagggcaccagcgccctgctgagcagcaaggtgaccaacatggtgctgagaaagagaaacaaggccagatacagccccctgcacaacgaggacgaggccggcgacgaggacgagctgtagaggagctagcgaattctgc

SEQ ID NO: 8 - DNA, 569 bp, Synthetic construct
  source 1..569: mol_type = other DNA
taatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtatataagcagagctcgtcgcatttgcacctcggcactcggagcgagacgcagcagccaggcagactcgggccgccccctctccgcatcaccacagaagccccgcctacgttgcgacccccaggg

SEQ ID NO: 9 - DNA, 58 bp, Synthetic construct
  source 1..58: mol_type = other DNA
tccctatcagtgatagagatctccctatcagtgatagagatcgtcgacgagctcgcgt

SEQ ID NO: 10 - DNA, 40 bp, Synthetic construct
  source 1..40: mol_type = other DNA
tccctatcagtgatagagatctccctatcagtgatagaga

SEQ ID NO: 11 - DNA, 19 bp, Synthetic construct
  source 1..19: mol_type = other DNA
tccctatcagtgatagaga

SEQ ID NO: 12-13 - skipped (2 entries)

SEQ ID NO: 14 - DNA, 953 bp, Synthetic construct
  source 1..953: mol_type = other DNA
atgggagaggcgtcgctgccggcccaggccgccgagacggaggaggtgggtcttttgtcgaaaaatacctccggtccgatgtcgcgccggcggaaattgtcgcgctcatgcgcaacctcaacagcctgatgggacgcacgcggtttatttacctggcgttgctggaggcctgtctccgcgttcccatggccacccgcagcagcgccatatttcggcggatctatgaccactacgccacgggcgtcatccccacgatcaacgtcaccggagagctggagctcgtggccctgccccccaccctgaacgtaacccccgtctgggagctgttgtgcctgtgcagcaccatggccgcgcgcctgcattgggactcggcggccgggggatctgggaggaccttcggccccgatgacgtgctggacctactgaccccccactacgaccgctacatgcagctggtgttcgaactgggccactgtaacgtaaccgacggacttctgctctcggaggaagccgtcaagcgcgtcgccgacgccctaagcggctgtcccccgcgcgggtccgttagcgagacggaccacgcggtggcgctgttcaagataatctggggcgaactgtttggcgtgcagatggccaaaagcacgcagacgtttcccggggcggggcgcgttaaaaacctcaccaaacagacaatcgtggggttgttggacgcccaccacatcgaccacagcgcctgccggacccacaggcagctgtacgccctgcttatggcccacaagcgggagtttgcgggcgcgcgcttcaagctacgcgtgcccgcgtgggggcgctgtttgcgcacgcactcatccagcgccaaccccaacgctgacatcatcctggaggcggcgctgtcggagctccccaccgaggcctggcccatgatgcagggggcggtgaactttagcaccctataa

SEQ ID NO: 15 - DNA, 1635 bp, Human alphaherpesvirus 2
  source 1..1635: mol_type = genomic DNA
cttgggggggggggggaagaaactaaaaacacatcaagcccacaacccatcccacaaggggggttatggcggacccaccgcaccaccatactccgattcgaccacatatgcaaccaaatcacccccagaggggaggttccatttttacgaggaggaggagtataatagagtctttgtgtttaaaacccggggtcggtgtggtgttcggtcataagctgcattgcgaaccactagtcgccgtttttcgtgtgcatcgcgtatcacggcatggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgttaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggcgctggtcatcggcggtattgcgttttgggtacgccgccgcgctcagatggcccccaagcgcctacgtctcccccacatccgggatgacgacgcgcccccctcgcaccagccattgttttactagaggagtttccccgttcccgtgtacctctgggcccgtgtgggagggtggccggggtatttgggtgggacttggactccgcataaagggagtctcgaaggagggaaactaggacagttcataggccgggagcgtggggcgcgcaccgcgtcccgacgattagccaccgcgcccacagccacctcgacc

SEQ ID NO: 16 - DNA, 618 bp, Human alphaherpesvirus 1
  source 1..618: mol_type = genomic DNA
taatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccc

SEQ ID NO: 17 - protein, 393 aa, Human alphaherpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 18 - protein, 901 aa, Human alphaherpesvirus 2
MRGGGLICALVVGALVAAVASAAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 19 - protein, 698 aa, Human alphaherpesvirus 2
MHAIAPRLLLLFVLSGLPGTRGGSGVPGPINPPNNDVVFPGGSPVAQYCYAYPRLDDPGPLGSADAGRQDLPRRVVRHEPLGRSFLTGGLVLLAPPVRGFGAPNATYAARVTYYRLTRACRQPILLRQYGGCRGGEPPSPKTCGSYTYTYQGGGPPTRYALVNASLLVPIWDRAAETFEYQIELGGELHVGLLWVEVGGEGPGPTAPPQAARAEGGPCVPPVPAGRPWRSVPPVWYSAPNPGFRGLRFRERCLPPQTPAAPSDLPRVAFAPQSLLVGITGRTFIRMARPTEDVGVLPPHWAPGALDDGPYAPFPPRPRFRRALRTDPEGVDPDVRAPLTGRRLMALTEDASSDSPTSAPEKTPLPVSATAMAPSVDPSAEPTAPATTTPPDEMATQAATVAVTPEETAVASPPATASVESSPLPAAAATPGAGHTNTSSAPAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPDDDDSATGLAFRTPNPNKPPPARPGPIRPTLPPGILGPLAPNTPRPPAQAPAKDMPSGPTPQHIPLFWFLTASPALDILFIISTTIHTAAFVCLVALAAQLWRGRAGRRRYAHPSVRYVCLPPERD

SEQ ID NO: 20 - protein, 851 aa, Human alphaherpesvirus 1
MPFVGGAESGDPLGAGRPIGDDECEQYTSSVSLARMLYGGDLAEWVPRVHPKTTIERQQHGPVTFPNASAPTARCVTVVRAPMGSGKTTALIRWLREAIHSPDTSVLVVSCRRSFTQTLATRFAESGLVDFVTYFSSTNYIMNDRPFHRLIVQVESLHRVGPNLLNNYDVLVLDEVMSTLGQLYSPTMQQLGRVDALMLRLLRTCPRIIAMDATANAQLVDFLCGLRGEKNVHVVVGEYAMPGFSARRCLFLPRLGTELLQAALRPPGPPSGPSPDASPDARGATFFGELEARLGGGDNICIFSSTVSFAEIVARFCRQFTDRVLLLHSLTPLGDVTTWGQYRVVIYTTVVTVGLSFDPLHFDGMFAYVKPMNYGPDMVSVYQSLGRVRTLRKGELLIYMDGSGARSEPVFTPMLLNHVVSSCGQWPAQFSQVTNLLCRRFKGRCDASACDTSLGRGSRIYNKFRYKHYFERCTLACLSDSLNILHMLLTLNCIRVRFWGHDDTLTPKDFCLFLRGVHFDALRAQRDLRELRCRDPEASLPAQAAETEEVGLFVEKYLRSDVAPAEIVALMRNLNSLMGRTRFIYLALLEACLRVPMATRSSAIFRRIYDHYATGVIPTINVTGELELVALPPTLNVTPVWELLCLCSTMAARLHWDSAAGGSGRTFGPDDVLDLLTPHYDRYMQLVFELGHCNVTDGLLLSEEAVKRVADALSGCPPRGSVSETDHAVALFKIIWGELFGVQMAKSTQTFPGAGRVKNLTKQTIVGLLDAHHIDHSACRTHRQLYALLMAHKREFAGARFKLRVPAWGRCLRTHSSSANPNADIILEAALSELPTEAWPMMQGAVNFSTL

SEQ ID NO: 21 - protein, 317 aa, Synthetic construct
MGEASLPAQAAETEEVGLFVEKYLRSDVAPAEIVALMRNLNSLMGRTRFIYLALLEACLRVPMATRSSAIFRRIYDHYATGVIPTINVTGELELVALPPTLNVTPVWELLCLCSTMAARLHWDSAAGGSGRTFGPDDVLDLLTPHYDRYMQLVFELGHCNVTDGLLLSEEAVKRVADALSGCPPRGSVSETDHAVALFKIIWGELFGVQMAKSTQTFPGAGRVKNLTKQTIVGLLDAHHIDHSACRTHRQLYALLMAHKREFAGARFKLRVPAWGRCLRTHSSSANPNADIILEAALSELPTEAWPMMQGAVNFSTL

SEQ ID NO: 22 - DNA, 259 bp, Human alphaherpesvirus 1
  source 1..259: mol_type = genomic DNA
caacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacc

SEQ ID NO: 23 - DNA, 302 bp, Human alphaherpesvirus 2
  source 1..302: mol_type = genomic DNA
gccgatccggcctcgggtctgcttgcccctcccccggcccagcacaggcaggctcgtccgacttccgcatacaccccaccctaccgcgtgcttccgcacccccgcctacgcgtgtacgcgaaggcggacccagacctgccgtatgctaattaaatacataaaacccaccctcggtgtccgattggtttctggggacggcgggggcgggggcggtgacgcccgacggggagggacaaggaggagtttcggaaagccggccccggtcgtgcgggtataagggcagccaccggcccactgggcgc

SEQ ID NO: 24 - DNA, 1974 bp, Synthetic construct
  source 1..1974: mol_type = other DNA
caattgaagcttcgtacggggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagccgagctctccctatcagtgatagagatctccctatcagtgatagagatcgtcgacgagctcgcgtgtgcatcgcgtatcacggcgccaccatgggccgcctgaccagcggcgtgggcaccgccgccctgctggtggtggccgtgggcctgcgcgtggtgtgcgccaagtacgccctggccgaccccagcctgaagatggccgaccccaaccgcttccgcggcaagaacctgcccgtgctggaccagctgaccgacccccccggcgtgaagcgcgtgtaccacatccagcccagcctggaggaccccttccagccccccagcatccccatcaccgtgtactacgccgtgctggagcgcgcctgccgcagcgtgctgctgcacgcccccagcgaggccccccagatcgtgcgcggcgccagcgacgaggcccgcaagcacacctacaacctgaccatcgcctggtaccgcatgggcgacaactgcgccatccccatcaccgtgatggagtacaccgagtgcccctacaacaagagcctgggcgtgtgccccatccgcacccagccccgctggagctactacgacagcttcagcgccgtgagcgaggacaacctgggcttcctgatgcacgcccccgccttcgagaccgccggcacctacctgcgcctggtgaagatcaacgactggaccgagatcacccagttcatcctggagcaccgcgcccgcgccagctgcaagtacgccctgcccctgcgcatcccccccgccgcctgcctgaccagcaaggcctaccagcagggcgtgaccgtggacagcatcggcatgctgccccgcttcatccccgagaaccagcgcaccgtggccctgtacagcctgaagatcgccggctggcacggccccaagcccccctacaccagcaccctgctgccccccgagctgagcgacaccaccaacgccacccagcccgagctggtgcccgaggaccccgaggacagcgccctgctggaggaccccgccggcaccgtgagcagccagatcccccccaactggcacatccccagcatccaggacgtggccccccaccacgcccccgccgcccccagcaaccccggcctgatcatcggcgccctggccggcagcaccctggccgtgctggtgatcggcggcatcgccttctgggtgcgccgccgcgcccagatggcccccaagcgcctgcgcctgccccacatccgcgacgacgacgccccccccagccaccagcccctgttctactaaaggagtttccccgctcccgtgtacctctgggcccgtgtgggagggtggctggggtatttgggtgggacttggactccgcataaagggagtctcgaaggagggaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgatatctacgcaacgaccccgcccatgggtcccaattggatcctctaga

SEQ ID NO: 25 - DNA, 2530 bp, Synthetic construct
  source 1..2530: mol_type = other DNA
tgccggccgcggggacggtggcctacggacaccccggcgccggcccgtccccgcactacccgcctcctcccgcccacccgtacccgggtatgctgttcgcgggccccagtcccctggaggcccagatcgccgcgctggtgggggccatcgccgccgaccgccaggcgggtgggcttccggcggccgccggagaccacgggatccgggggtcggcgaagcgccgccgacacgaggtggagcagccggagtacgactgcggccgtgacgagccggaccgggacttcccgtattacccgggcgaggcccgccccgagccgcgcccggtcgactcccggcgcgccgcgcgccaggcttccgggccccacgaaaccatcacggcgctggtgggggcggtgacgtccctgcagcaggaactggcgcacatgcgcgcgcgtacccacgccccctacgggccgtatccgccggtggggccctaccaccacccccacgcagacacggagacccccgcccaaccaccccgctaccccgccaaggccgtctatctgccgccgccgcacatcgcccccccggggcctcctctatccggggcggtccccccaccctcgtatcccccagttgcggttacccccggtcccgctcccccgctacatcagccctcccccgcacacgcccacccccctccgccgccgccgggacccacgcctccccccgccgcgagcttaccccaacccgaggcgcccggcgcggaggccggcgccttagttaacgccagcagcgcggcccacgtgaacgtggacacggcccgggccgccgatctgtttgtgtcacagatgatggggtcccgctaactcgcctccaggatccggacttggggggggtgtgtgttttcatatattttaaataaacaaacaaccggacaaaagtatacccacttcgtgtgcttgtgtttttgtttgagaggggggggtggagtgggggggaaagtgggccgaatgacacaaaaattaggtcgtacgcaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtatataagcaaccggtgtcgacggcgggggtcgtcggggtccgtgggtctcgccccctccccccatcgagagtccgtaggtgacctaccgttgcgccaccaccagaggccatatccgacaccccagccccgacggcagccgacagcccgaagcttggtcgatatctatcggagctgtgctagcgccactggtacccgagcggaattctgtctagaaatggttacaaataaaagatctttattttcattagatctgtgtgttggttttttgtgtgttggttttcccgttagcacatgtctgcatttgtttttctagtcacacgccccccccccccaaataaaaaccaaggcaaaacaataccagaagtcatgtgtatttttgaacatcggtgtctttttatttatacacaagcccagctcccctcccctcccttagagctcgtcttcgtctccggcctcgtcctcgttgtggagcggagagtacctggctttgttgcgcttgcgcagaaccatgttggtgaccttggagctgagcagggcgctcgtgcccttctttctggccttgtgttccgtgcgctccatggccgacaccaaagccatatatcggatcatttctcgggcctcggccaacttggcctcgtcaaacccgcccccctccgcgccttcctccccctccccgcccacgcccccggggtcggaagtcttgagttccttggtggtgagcggatacagggccttcatgggattgcgttgcagttgcaggacgtagcggaaggcgaagaaggccgcgaccaggccggccaggaccagcagccccacggcaagcgccccgaaggggttggacataaaggaggacacgcccgagacggccgacaccacgccccccactactcccatgactaccttgccgaccgcgcgccccaagtcccccatcccctcgaagaacgcgcacagccccgcgaacatggcggcgttggcgtcggcgcggatgaccgtgtcgatgtcggcaaagcgcaggtcgtgcagctggttgcggcgctggacctccgtgtagtccagcaggccgctgtccttgatctcgtggcgcgtgtagacctccaggggcacaaactcgtggtcctccagcatggtgatgttcaggtcgatgaaggtgctgacggtggtgacgtcggcgcgactcagctggtgagagtacgcgtactcctcgaagtacacgtagcccccgccgaagatgaagtagcgccggtggcccacggtgcacggctcgagcgcgtc

SEQ ID NO: 26 - DNA, 1010 bp, Human alphaherpesvirus 2
  source 1..1010: mol_type = genomic DNA
tgccggccgcggggacggtggcctacggacaccccggcgccggcccgtccccgcactacccgcctcctcccgcccacccgtacccgggtatgctgttcgcgggccccagtcccctggaggcccagatcgccgcgctggtgggggccatcgccgccgaccgccaggcgggtgggcttccggcggccgccggagaccacgggatccgggggtcggcgaagcgccgccgacacgaggtggagcagccggagtacgactgcggccgtgacgagccggaccgggacttcccgtattacccgggcgaggcccgccccgagccgcgcccggtcgactcccggcgcgccgcgcgccaggcttccgggccccacgaaaccatcacggcgctggtgggggcggtgacgtccctgcagcaggaactggcgcacatgcgcgcgcgtacccacgccccctacgggccgtatccgccggtggggccctaccaccacccccacgcagacacggagacccccgcccaaccaccccgctaccccgccaaggccgtctatctgccgccgccgcacatcgcccccccggggcctcctctatccggggcggtccccccaccctcgtatcccccagttgcggttacccccggtcccgctcccccgctacatcagccctcccccgcacacgcccacccccctccgccgccgccgggacccacgcctccccccgccgcgagcttaccccaacccgaggcgcccggcgcggaggccggcgccttagttaacgccagcagcgcggcccacgtgaacgtggacacggcccgggccgccgatctgtttgtgtcacagatgatggggtcccgctaactcgcctccaggatccggacttggggggggtgtgtgttttcatatattttaaataaacaaacaaccggacaaaagtatacccacttcgtgtgcttgtgtttttgtttgagaggggggggtggagtgggggggaaagtgggccgaatgacacaaaaattaggt

SEQ ID NO: 27 - DNA, 1000 bp, Human alphaherpesvirus 2
  source 1..1000: mol_type = genomic DNA
ttggttttcccgttagcacatgtctgcatttgtttttctagtcacacgccccccccccccaaataaaaaccaaggcaaaacaataccagaagtcatgtgtatttttgaacatcggtgtctttttatttatacacaagcccagctcccctcccctcccttagagctcgtcttcgtctccggcctcgtcctcgttgtggagcggagagtacctggctttgttgcgcttgcgcagaaccatgttggtgaccttggagctgagcagggcgctcgtgcccttctttctggccttgtgttccgtgcgctccatggccgacaccaaagccatatatcggatcatttctcgggcctcggccaacttggcctcgtcaaacccgcccccctccgcgccttcctccccctccccgcccacgcccccggggtcggaagtcttgagttccttggtggtgagcggatacagggccttcatgggattgcgttgcagttgcaggacgtagcggaaggcgaagaaggccgcgaccaggccggccaggaccagcagccccacggcaagcgccccgaaggggttggacataaaggaggacacgcccgagacggccgacaccacgccccccactactcccatgactaccttgccgaccgcgcgccccaagtcccccatcccctcgaagaacgcgcacagccccgcgaacatggcggcgttggcgtcggcgcggatgaccgtgtcgatgtcggcaaagcgcaggtcgtgcagctggttgcggcgctggacctccgtgtagtccagcaggccgctgtccttgatctcgtggcgcgtgtagacctccaggggcacaaactcgtggtcctccagcatggtgatgttcaggtcgatgaaggtgctgacggtggtgacgtcggcgcgactcagctggtgagagtacgcgtactcctcgaagtacacgtagcccccgccgaagatgaagtagcgccggtggcccacggtgcacggctcgagcgcgtc

SEQ ID NO: 28 - DNA, 514 bp, Synthetic construct
  source 1..514: mol_type = other DNA
caacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtatataagcaaccggtgtcgacggcgggggtcgtcggggtccgtgggtctcgccccctccccccatcgagagtccgtaggtgacctaccgttgcgccaccaccagaggccatatccgacaccccagccccgacggcagccgacagcccgaagcttggtcgatatctatcggagctgtgctagcgccactggtacccgagcggaattctgtctagaaatggttacaaataaaagatctttattttcattagatctgtgtgttggttttttgtgtg

SEQ ID NO: 29 - DNA, 521 bp, Synthetic construct
  source 1..521: mol_type = other DNA
cgtacgcaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtatataagcaaccggtctccctatcagtgatagagatctccctatcagtgatagagatcgaccggtgtcgacggcgggggtcgtcggggtccgtgggtctcgccccctccccccatcgagagtccgtaggtgacctaccgttgcgccaccaccagaggccatatccgacaccccagccccgacggcagccgacagcccgaagcttggtcgatatctatcggagctgtgctagcgccactggtacccgagcggaattctgtctagaaatggttaca

SEQ ID NO: 30 - DNA, 259 bp, Human alphaherpesvirus 1; identical to SEQ ID NO: 22
  source 1..259: mol_type = genomic DNA

SEQ ID NO: 31 - DNA, 58 bp, Human alphaherpesvirus 1
  source 1..58: mol_type = genomic DNA
tgcgccaccaccagaggccatatccgacaccccagccccgacggcagccgacagcccg

SEQ ID NO: 32 - DNA, 75 bp, Human alphaherpesvirus 1
  source 1..75: mol_type = genomic DNA
gtcgacggcgggggtcgtcggggtccgtgggtctcgccccctccccccatcgagagtccgtaggtgacctaccgt

SEQ ID NO: 33 - DNA, 66 bp, Synthetic construct
  source 1..66: mol_type = other DNA
aagcttggtcgatatctatcggagctgtgctagcgccactggtacccgagcggaattctgtctaga

SEQ ID NO: 34 - DNA, 49 bp, Synthetic construct
  source 1..49: mol_type = other DNA
aataaaagatctttattttcattagatctgtgtgttggttttttgtgtg

SEQ ID NO: 35 - DNA, 7841 bp, Synthetic construct
  source 1..7841: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgtgccggccgcggggacggtggcctacggacaccccggcgccggcccgtccccgcactacccgcctcctcccgcccacccgtacccgggtatgctgttcgcgggccccagtcccctggaggcccagatcgccgcgctggtgggggccatcgccgccgaccgccaggcgggtgggcttccggcggccgccggagaccacgggatccgggggtcggcgaagcgccgccgacacgaggtggagcagccggagtacgactgcggccgtgacgagccggaccgggacttcccgtattacccgggcgaggcccgccccgagccgcgcccggtcgactcccggcgcgccgcgcgccaggcttccgggccccacgaaaccatcacggcgctggtgggggcggtgacgtccctgcagcaggaactggcgcacatgcgcgcgcgtacccacgccccctacgggccgtatccgccggtggggccctaccaccacccccacgcagacacggagacccccgcccaaccaccccgctaccccgccaaggccgtctatctgccgccgccgcacatcgcccccccggggcctcctctatccggggcggtccccccaccctcgtatcccccagttgcggttacccccggtcccgctcccccgctacatcagccctcccccgcacacgcccacccccctccgccgccgccgggacccacgcctccccccgccgcgagcttaccccaacccgaggcgcccggcgcggaggccggcgccttagttaacgccagcagcgcggcccacgtgaacgtggacacggcccgggccgccgatctgtttgtgtcacagatgatggggtcccgctaactcgcctccaggatccggacttggggggggtgtgtgttttcatatattttaaataaacaaacaaccggacaaaagtatacccacttcgtgtgcttgtgtttttgtttgagaggggggggtggagtgggggggaaagtgggccgaatgacacaaaaattaggtcgtacgcaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtatataagcaaccggtgtcgacggcgggggtcgtcggggtccgtgggtctcgccccctccccccatcgagagtccgtaggtgacctaccgttgcgccaccaccagaggccatatccgacaccccagccccgacggcagccgacagcccgagcttaccatggggggttctcatcatcatcatcatcatggtatggctagcatgactggtggacagcaaatgggtcgggatctgtacgacgatgacgataaggtacctaaggatcagcttggagttgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggagttccagctgagcgccggtcgctaccattaccagttggtctggtgtcaaaaataataaagccgaattctgtctagaaatggttacaaataaaagatctttattttcattagatctgtgtgttggttttttgtgtgttggttttcccgttagcacatgtctgcatttgtttttctagtcacacgccccccccccccaaataaaaaccaaggcaaaacaataccagaagtcatgtgtatttttgaacatcggtgtctttttatttatacacaagcccagctcccctcccctcccttagagctcgtcttcgtctccggcctcgtcctcgttgtggagcggagagtacctggctttgttgcgcttgcgcagaaccatgttggtgaccttggagctgagcagggcgctcgtgcccttctttctggccttgtgttccgtgcgctccatggccgacaccaaagccatatatcggatcatttctcgggcctcggccaacttggcctcgtcaaacccgcccccctccgcgccttcctccccctccccgcccacgcccccggggtcggaagtcttgagttccttggtggtgagcggatacagggccttcatgggattgcgttgcagttgcaggacgtagcggaaggcgaagaaggccgcgaccaggccggccaggaccagcagccccacggcaagcgccccgaaggggttggacataaaggaggacacgcccgagacggccgacaccacgccccccactactcccatgactaccttgccgaccgcgcgccccaagtcccccatcccctcgaagaacgcgcacagccccgcgaacatggcggcgttggcgtcggcgcggatgaccgtgtcgatgtcggcaaagcgcaggtcgtgcagctggttgcggcgctggacctccgtgtagtccagcaggccgctgtccttgatctcgtggcgcgtgtagacctccaggggcacaaactcgtggtcctccagcatggtgatgttcaggtcgatgaaggtgctgacggtggtgacgtcggcgcgactcagctggtgagagtacgcgtactcctcgaagtacacgtagcccccgccgaagatgaagtagcgccggtggcccacggtgcacggctcgagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 36 - DNA, 4389 bp, Synthetic construct
  source 1..4389: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggcgcgccgccgcgcgggcccggactccgccccggcgaccgccccgcgccggcttcccggtatggtaattagaaacttttaataggcggtcccggccgccatccccgctcatggcaattagcaacttttaatgggccggccttcccgctcgcggtaattagcagcttttaacgggccgccattcccgcttatggtaattaaaaacgttcggacggcccctcgctccccgcgtaattactccctcggggttccgggttatgctgattactttcttggcagaacacgcagagcctcgcgcgccgccgggtgggtgggctgatcggcccctattggtcccctgggcttcctagtatgctaatgaatttctccccgggggcgggcaccactcagggccgcgccggcggggcgccggggggactcccatctgcgtcggcgggggcggcgcatgctaatggggttcttggagtacacccggttggtccccggggacggggccgccccgagagggggggattccctccctccgcccccgccggggcgcgcggctattgggggaatcgtaaatgccgcccctttgggggagtggataggcgccgggtataaggcagccccgtgtgacggtcgggccgcattcgcaccccggcactgcgagcgacggagcggcggcccggcgggaggaggagacccggagagacagagactaaaacccggcaagagagagagaccgcgggccgccgtctcgaagcttggtcgatatctatcggagctgtgctagcgccactggtacccgagcggaattcggcttgctgcccgaagggaagccgcccccccccggaccaccggccgaggcgcctcgggggcagggggaggtgggggggggaagacggggaggagacaggaagtgggggtgggagtggggggggacggacacggccccgaacagcaacacacaccagcattttgttatggactttctggccttgttgaaaacttgaggaaaaaaaactttatatttataaaaattttacaataaagttttgtgatgcttttgacacactttgttgttggcctttgatgcagctcccccgcgcaggggggccggggatgggggggaaagggaggaggaggagggggggcgggcacgagaagccgcccccacccccgaggcctgttggtctttatcatagaacagagccggggcccggcctgcgttctggctccctgtcttggtgggtgggcgggctggctggcgggtaaaaaaagagtgtgtccgtgttgacagggaggggggcccgatcgtgcagagcacgcacgtctggccggccagaccctgggggtggtgggcaggagtgggagggcgcctggctcggggagggaggagggggggggtcagccgcaccaccggcgcgaagccaggggccagggaactttgatagagaggggggaaagtggggcgggggcgagggcggttgaatcacaacgcatgcacgccctctgcccccggggacgggtgggaggaaggaggagggagaagagaagacccgaggcatgcacccgcacttacgcccgtgcccacccccgccccggcgcccaccccgcccgcacacctgcccgccacgcccgcccctcctcaccctggctgggagaaaggaggaggagcaggaagaggagacccgaggcatgcaaccgcactcaccccaccccgcccgcacacctgcccgccacgcccgcccctccttaccctggctgcggggagactcccatcggggcgagggggctcgcgcgttcgcaacaccacaccacaccacacggcccaccacaacacggcccaccacgacacaacacgacacgacgcgttttgcggggcatgcaagtcgacacaccgcgcgcgtgcctacctttccctagcggccccggccccccggcccgtttccttccgccaccactaccaccacccccccgcccgcgcccacgcggtagaggaaggggacgggcgccacacccacggctgtggccgggcacgcgcctttggggttgttgggggggggtgaccggcgcgtgggggcggtgggcgtatgggcccgacccgcgcctgccccccctgggaacgacgcccccggacgacaccacgggggggggaaacgggggtgggtggaagggaagaggaaggagaaagggggggtggatccgaacacgccggactcgagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 37 - DNA, 396 bp, Synthetic construct
  source 1..396: mol_type = other DNA
  misc_feature 304..311: note = TATA element
gggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagccgagctctccctatcagtgatagagatctccctatcagtgatagagatcgtcgacgagctcgcgtgtgcatcgcgtatcacggc

(19) United States
(12) Patent Application Publication
(10) Pub. No.: US 2025/0367279 A1
(43) Pub. Date: Dec. 4, 2025

(54) RECOMBINANT HERPES SIMPLEX VIRUS-2 EXPRESSING GLYCOPROTEIN D AND B ANTIGENS

(71) Applicant: The Brigham and Women's Hospital, Inc., Boston, MA (US)

(72) Inventor: Feng Yao, Southborough, MA (US)

(21) Appl. No.: 18/873,079

(22) PCT Filed: Jun. 9, 2023
(86) PCT No.: PCT/US2023/068169

§ 371 Date: Dec. 9, 2024

Related U.S. Application Data
(60) Provisional application No. 63/350,760, filed on Jun. 9, 2022.

(51) Int. Cl. A61K 39/245; A61K 39/00; A61P 31/22; C07K 14/005; C12N 7/00

(52) U.S. Cl. CPC A61K 39/245; A61P 31/22; C07K 14/005; C12N 7/00; A61K 2039/5254; C12N 2710/16621; C12N 2710/16622; C12N 2710/16634; C12N 2710/16652

(57) ABSTRACT

Provided herein are recombinant Herpes Simplex Virus-2 comprising sequences encoding glycoprotein D and B antigens, with two sequences encoding dominant negative UL9 proteins, compositions comprising the same, and methods of use thereof.

### CLAIM OF PRIORITY

This application claims the benefit of U.S. Provisional Patent Application Ser. No. 63/350,760, filed on Jun. 9, 2022. The entire contents of the foregoing are hereby incorporated by reference.

### SEQUENCE LISTING

This application contains a Sequence Listing that has been submitted electronically as an XML file named 29618-0299WO1_SL_ST26.xml. The XML file, created on Jun. 9, 2023, is 68,384 bytes in size. The material in the XML file is hereby incorporated by reference in its entirety.

### FIELD OF THE INVENTION

Provided herein are recombinant Herpes Simplex Virus-2 comprising sequences encoding glycoprotein D and B antigens, with two sequences encoding dominant negative UL9 proteins, compositions comprising the same, and methods of use thereof.

### BACKGROUND

Genital herpes is one of the most common sexually transmitted diseases worldwide and is the primary cause of genital ulcer disease in both developed and developing countries. Herpes simplex virus type 2 (HSV-2) is the primary cause of recurrent genital herpes. Approximately fifty to sixty million adults in the United States are infected with HSV-2. HSV infections can cause significant clinical problems in AIDS and cancer patients, organ transplant recipients, and newborns. Moreover, genital HSV-2 infection triples the risk for sexual acquisition and transmission of HIV infection. Currently, no antiviral therapy is effective in preventing or reducing the incidence of symptomatic and asymptomatic HSV-2 recurrent infections with the exception of daily suppressive therapy. Thus, there is a strong need to develop safe and efficacious vaccines against HSV-2 infection.

It is well documented that live viral vaccines capable of de novo synthesis of immunogens in the host induce a broader and more durable immune response than vaccines consisting of only peptides or proteins. Various forms of replication-defective HSV and neuro-attenuated, replication-competent mutants have been developed and tested as potential vaccines against HSV infection. However, because both replication-defective viruses and neuro-attenuated mutants can co-replicate with wild-type virus or become replication-competent in the context of wild-type virus, their use as a vaccine in humans poses a safety concern, particularly in individuals who harbor latent HSV infection. The observation that replication-defective HSV-1 mutants can reactivate the latent HSV-1 immediate-early promoter in the rodent brain has raised additional safety concerns about the possibility of such recombinants triggering outbreaks of productive viral infections in latently infected individuals.

### SUMMARY

The CJ2-gD2/gB2 HSV-2 recombinant viral vaccine (described in WO2019152821) was engineered using tetracycline gene-switch technology (T-REX, Invitrogen, Yao et al., Hum. Gene Ther. 9:1939-50 (1998)) and a dominant-negative mutant form of the HSV-1 UL9 polypeptide, e.g., UL9-C535C, to develop a safe and effective recombinant viral vaccine against HSV-2 infection. Exemplary methods for making and using HSV-2 vaccines based on this technology can be found in US 2012/0263752 (incorporated herein by reference in its entirety).

Described herein are improved HSV-2 recombinant viral vaccines, one example of which is referred to herein as CJVAC, comprising dominant-negative Herpes simplex virus-2 (HSV-2) recombinant virus. The genome of the virus has a first coding sequence that codes for HSV-2 glycoprotein B (gB2) and which is operably linked to a first promoter that is under the control of (operably linked to) a first tetracycline operator (tet-O) nucleotide segment. In these recombinant virus an HSV-2 non-essential gene, e.g., the gG2 gene, is replaced by a gB2 insertion. One advantage of doing this is that because deletion of gG2 gene has no effect on HSV-2 viral replication (Liljeqvist et al, J Virol. 73:9796-802 (1999); Harland et al, J Gen. Virol. 69 (Pt 1): 113-24 (1988)), one can produce gG2 deletion mutant virus in cells that do not express gG2. An additional advantage will be that assays, e.g., by PCR or serological tests, can be performed to determine whether gG2 is being expressed in the vaccinated host in order to differentiate between infection with wild type HSV-2 and with the vaccine vector. This ability may be important in serological differentiation for breakthrough HSV-2 infections in clinical settings. For comparison, a virus in which gB2 is inserted at a genomic site that is important for virus replication, the UL9 locus, is also described.

The recombinant viruses, such as CJVAC, described herein include a) a sequence comprising a codon optimized HSV-2 glycoprotein B2 (gB2) coding sequence operably linked to an ICP0 promoter that is operably linked to a tetO sequence and optionally part of a 5′ untranslated region of ICP0 gene, wherein the gB2 coding sequence is inserted at the gG2 locus of the HSV-2 genome; b) a sequence comprising a codon-optimized HSV-2 glycoprotein D2 (gD2) coding sequence operably linked to an HSV-1 or HSV-2 immediate early promoter, wherein the gD2 coding sequence is inserted at the ICP0 locus (i.e., two gD2 coding sequences, one inserted in the ICP0 locus in each of the TRL and IRL); c) a sequence comprising a first coding sequence encoding a dominant negative mutant HSV-1 or HSV-2 UL9 protein (dnUL9) operably linked to a ICP27 promoter that is operably linked to a tetO sequence and a codon-optimized gD2 sequence operably linked to an ICP4 promoter that is operably linked to a tetO sequence, wherein the dnUL9/gD2 sequence is inserted into the intergenic region of the HSV-2 UL26 and UL27 genes; and d) a sequence comprising a second dnUL9 coding sequence operably linked to an ICP4 promoter that is operably linked to a tetO sequence wherein the second UL9-C535C coding sequence is inserted at the gD2 locus. Preferably, the genome of the virus does not comprise a sequence encoding a functional endogenous ICP0 protein or a functional endogenous HSV-2 gG2 protein. The dominant negative mutant HSV-1 or HSV-2 UL9 protein, most preferably UL9-C535C, is used to control viral replication. When this sequence is expressed, the mutant protein produced acts in trans to inhibit the replication of HSV-2. Each of the tet operators allows transcription to proceed from its associated promoter when free of tet repressor but leads to the blocking of transcription when bound by repressor. In order to enhance its antigenicity, the genome may also express recombinant immunomodulating genes, such as IL-12, IL-15 or may express other HSV-1 or HSV-2 major antigens in levels comparable to, or higher than, the levels expressed by wild type HSV-1 or HSV-2 viruses.

Thus, provided herein are replication-defective Herpes simplex virus 2 (HSV-2) recombinant virus, comprising within its genome: a) a sequence comprising a codon optimized HSV-2 glycoprotein B2 (gB2) coding sequence operably linked to an ICP0 promoter that is operably linked to a tetO sequence, wherein the gB2 coding sequence is inserted at the gG2 locus of the HSV-2 genome; b) a sequence comprising a codon-optimized HSV-2 glycoprotein D2 (gD2) coding sequence operably linked to an HSV-1 or HSV-2 immediate early promoter, wherein the gD2 coding sequence is inserted at the ICP0 locus; c) a sequence comprising a first coding sequence encoding a dominant negative mutant HSV-1 or HSV-2 UL9 protein (dnUL9) operably linked to a ICP27 promoter that is operably linked to a tetO sequence and a codon-optimized gD2 sequence operably linked to an ICP4 promoter that is operably linked to a tetO sequence, wherein the dnUL9/gD2 sequence is inserted into the intergenic region of the HSV-2 UL26 and UL27 genes; and d) a sequence comprising a second dnUL9 coding sequence operably linked to an ICP4 promoter that is operably linked to a tetO sequence wherein the second UL9-C535C coding sequence is inserted at the gD2 locus, wherein the genome of the virus does not comprise a sequence encoding a functional endogenous ICP0 protein, a functional endogenous HSV-2 gG2 protein.

In some embodiments, the genome of the virus does not comprise an endogenous sequence encoding a functional HSV-2 gD2 protein (preferably encodes a gD2 gene under an HSV IE promoter at a different region in the HSV-2 genome).

In some embodiments, any one or more of the gB2, gD2, UL9-C535C/gD2, and UL9-C535C coding sequences are codon optimized.

In some embodiments, the ICP0 promoter that is operably linked to a tetO sequence in part a) comprises SEQ ID NO:8.

In some embodiments, the HSV-1 or HSV-2 immediate early promoter in b) is selected from the group consisting of an ICP0 promoter, an ICP27 promoter, and an ICP4 promoter.

In some embodiments, the HSV-1 or HSV-2 immediate early promoter in b) is a HSV-1 or HSV-2 ICP0 promoter.

In some embodiments, the dominant negative mutant HSV-1 or HSV-2 UL9 protein is UL9-C535C.

Also provided herein are vaccines comprising a recombinant virus as described herein.

Additionally provided are methods of immunizing a subject against HSV-1 or HSV-2 infection (i.e., to reduce the risk of developing the infection), or treating an HSV-1 or HSV-2 infection in a subject; the methods comprise administering to the subject a vaccine, virus, or composition as described herein. Also provided herein are viruses as described herein, compositions comprising a recombinant virus as described herein, and vaccines as described herein for use in the treatment or prophylaxis of an infection with HSV-1 or HSV-2.

In some embodiments, the subject is seropositive for HSV-1. In some embodiments, the subject is seropositive for HSV-2. In some embodiments, the subject is seronegative for HSV-1 and HSV-2. The methods can be used to reduce risk, severity, or frequency of recurrent HSV-2 and HSV-1 infection in HSV-1 and HSV-2 sera positive subjects.

Further, provided herein are methods for producing a virus as described herein. In some embodiments, the methods can include: a) infecting complementing cells with the virus, wherein the complementing cells express a functional gene product or products that are needed for replication of the virus and for which sequences encoding such are lacking from the virus genome; b) culturing the complementing cells such that the virus replicates; and c) harvesting the replicated virus from the complementing cells.

In some embodiments, the complementary cells further express TetR.

In some embodiments, the complementary cells express ICP0O functional gene product.

Described herein are recombinant herpes simplex virus-2 in accordance with any of the disclosed embodiments, methods of synthesizing the recombinant herpes simplex virus-2, and methods of using the recombinant herpes simplex virus-2.

### DESCRIPTION OF THE DRAWINGS

Some embodiments of the disclosure are described herein with reference to the accompanying figures. The description, together with the figures, make apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIG. 1 is a schematic drawing of the genomes of a non-replicating dominant-negative HSV-2 recombinant viral vaccine candidates, CJ2-gD2 and CJ2-gD2/gB2. The HSV-2 genome includes unique long (UL) and unique short (US) regions. Each of the UL and US is flanked by inverted copies of large repeats, the terminal and internal repeats of the long region (TRL/IRL) and the short region (TRS/IRS) (Szpara et al., J Virol. 2014 January; 88 (2): 1209-1227). CJ2-gD2/gB2 is a derivative of CJ2-gD2, in which the HSV-2 gG2 coding sequence in CJ2-gD2 is replaced with a codon-optimized gB2 sequence under control of the tetO-bearing modified HSV-1 ICP0 promoter plus part of 5′ untranslated region of ICP0 gene. The replacements of both copies of the ICP0 coding sequences with DNA sequences encoding UL9-C535C under control of the tetO-bearing hCMV major immediate-early promoter and gD2 under the tetO-bearing HSV-1 ICP4 promoter in CJ2-gD2 are shown expanded above the ICP0 coding sequences of the HSV-2 genome, while replacement of gG2 with the codon-optimized gB2 gene is shown expanded below the gG2 gene.

FIG. 2 is a schematic drawing of the genome of a non-replicating dominant-negative HSV-2 recombinant virus, CJOD-lacZ-NF87, in which 1) the HSV-2 gG2 coding sequence is replaced with a codon-optimized gB2 sequence under control of the tetO-bearing modified HSV-1 ICP0 promoter plus part of 5′ untranslated region of ICP0 gene, 2) the ICP0 gene at the ICP0 locus is replaced with the codon-optimized gD2 gene under the control of HSV-2 ICP0 promoter, 3) a new UL9-C535C/gD2 expression cassette is inserted into the intergenic region of UL26 and UL27 genes, which encodes UL9-C535C under the control of the tetO-containing HSV-1 ICP27 promoter (ICP27TO) and the codon-optimized gD2 under the control of the tetO-containing HSV-1 ICP4 promoter (ICP4TO), and 4) the HSV-2 gD2 gene at the gD2 locus is replaced with the lacZ gene.

FIG. 3 shows the plaque-forming efficiency of different passages of CJ2-gD2/gB2 and CJOD-lacZ-NF87 in VOR-124 cells in the absence and presence of doxycycline. The number on the top of each column represents fold of reduction in viral titer in the presence of doxycycline compared with in the absence of doxycycline in standard plaque assays.

FIG. 4 is a schematic drawing of the genome of an exemplary new generation non-replicating dominant-negative HSV-2 recombinant viral vaccine candidate, CJVAC, in which the HSV-2 gG2 coding sequence is replaced with a codon-optimized gB2 sequence under control of the tetO-bearing modified HSV-1 ICP0 promoter plus part of 5′ untranslated region of ICP0 gene as described for CJ2-gD2/gB2, the UL9-C535C/gD2 sequence in CJ2-gD2/gB2 at the ICP0 locus is replaced with the codon-optimized gD2 gene under the control of HSV-2 ICP0 promoter, a new UL9-C535C/gD2 expression cassette, which encodes UL9-C535C under the control of the ICP27TO and the codon-optimized gD2 under the control of the ICP4TO, is inserted into the intergenic region of the HSV-2 UL26 and UL27 genes, and the UL9-C535C gene under the control of the modified tetO-containing HSV-1 ICP4 promoter at the gD2 locus.

FIG. 5 shows that CJVAC expresses gD2 and gB2 as efficiently as CJ2-gD2/gB2 in Vero cells. Vero cells in duplicate were either mock-infected or infected with CJ2-gD2/gB2 or CJVAC at an MOI of 2 PFU/cell. Infected cell extracts were prepared at 20 h post-infection. Proteins in infected cell extracts were resolved on SDS-PAGE, followed by immunoblotting with monoclonal antibodies against gB2, HSV-1/2 gD, or ICP27 (HSV-specific input control).

FIG. 6 shows that CJVAC encodes fusogenic activity. VOR-124 cells were seeded at 5×10e5 cells per 60 mm dish. At 70 h post cells seeding, cells were either mock-infected or infected with CJVAC and CJ2-gD2/gB2 at an MOI of 0.02 PFU/cell. Photos were taken at 48 h post-infection.

FIG. 7 shows CJVAC replicates more efficiently than CJ2-gD2-gB2 in VOR-124 cells. VOR-124 cells were seeded at 4×10e5 cells per 60 mm dish. At 68 hours post seeding, cells in multiple dishes were infected with either CJ2-gD2/gB2 or CJVAC at a MOI of 0.02 PFU/cell. Infections were carried out at 34 C. Infected cells were harvested at about 69 hours post-infection and viral titers were determined by standard plaque assay on VOR-124 cell monolayers. Number on the top of the graph indicates the fold of difference in viral titer between CJVAC and CJ2-gD2/gB2.

FIG. 8 shows a trans-dominant-negative effect of CJVAC on replication of wild-type HSV-2. Vero cells were infected in triplicate with either wild-type HSV-2 strain 186 alone at an MOI of 2 PFU/cell; with wild-type HSV-2 (MOI 2) and N2-lacZ, a HSV-2 ICP0 deletion mutant that does not express UL9-C535C, at an MOI of 5 PFU/cell; with wild-type HSV-2 (MOI 2) and CJVAC at an MOI of 5 PFU/cell. Infected cells were harvested at 18 h post-infection and virus titers were determined on Vero cell monolayers. Viral titers are expressed as the mean+/−SD. Number on the top of the graph indicates the fold reduction in wild-type virus yield between single infection and co-infection.

### DETAILED DESCRIPTION

Using the T-REx gene switch technology (Invitrogen Inc., CA; Yao et al., Hum. Gene Ther. 9:1939-50 (1998)) and the dominant-negative mutant polypeptide UL9-C535C of the HSV-1 origin of viral replication binding protein UL9, a novel class of HSV-1 recombinants capable of inhibiting wild-type HSV-1 and HSV-2 infections (dominant-negative) was constructed. CJ83193 is a first generation HSV-1 recombinant virus that encodes UL9-C535C under the control of the tetracycline operator (tetO)-bearing hCMV major immediate-early promoter (CMVTO). CJ83193 expresses high levels of UL9-C535C in non-tetracycline repressor (tetR)-expressing cells, leading to inhibition of its own viral DNA replication and that of wild-type HSV-1 and HSV-2 in co-infected cells (Yao and Eriksson, Hum Gene Ther 10:1811-8, 1999; Antiviral Res 53:127-33, 2002). CJ9-gD is a CJ83193 derived HSV-1 recombinant viral vaccine candidate that encodes UL9-C535C and an extra copy of the HSV-1 glycoprotein D (gD) gene driven by the CMVTO promoter (Lu et al., J Invest Dermatol 129:1174-1184, 2009). CJ9-gD is completely replication-defective, cannot establish detectable latent infection in vivo, and expresses high levels of HSV-1 major antigen glycoprotein D (gD) independent of HSV-1 viral DNA replication (Lu et al., J Invest Dermatol 129:1174-1184, 2009). Immunization with CJ9-gD elicits a strong and long-term protective immune response against HSV-1 infection in mouse and guinea pig models of HSV-1 infections (Brans et al., J Invest Dermatol 128:2825-2832, 2008; Lu et al., J Invest Dermatol 129:1174-1184, 2009; Brans et al., J Invest Dermatol 129:2470-2479, 2009).

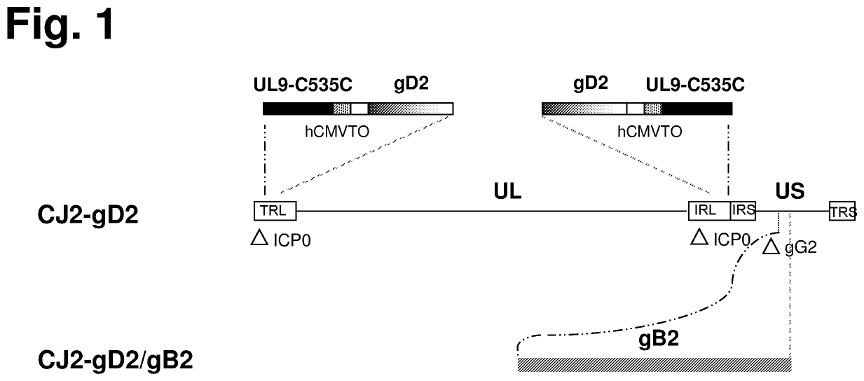

CJ2-gD2 is an HSV-2 ICP0 deletion mutant based non-replicating and dominant-negative recombinant virus, in which both copies of the HSV-2 ICP0 gene are replaced with a bi-directional transcription unit that encodes the full-length gD2 gene driven by the tetO-bearing HSV-1 ICP4 promoter and the UL9-C535C gene under the control of CMVTO (U.S. Pat. No. 8,809,047) (FIG. 1). CJ2-gD2 is avirulent and incapable of establishing detectable latent infection following immunization. High-level expression of gD2 by CJ2-gD2 lead to a significant increase in its efficacy in eliciting anti-HSV-2-specific neutralizing antibody response and protective immunity against wild-type HSV-2 genital infection and disease in mice compared with a non-gD2-expressing non-replicating dominant-negative HSV-2 recombinant virus (Akhramayeva et al, J. Virol. 85 (10): 5036-5047). Moreover, CJ2-gD2 was vastly superior to gD2-alum/MPL subunit vaccine in protection against HSV-2 genital infection in guinea pigs (Zhang et al., PLOS One 9: e101373, 2014).

To further enhance the vaccine efficacy of CJ2-gD2 and enable serological testing its vaccine efficacy in the vaccinated cohorts, a CJ2-gD2-derived vaccine construct, named CJ2-gD2/gB2, was constructed by replacing the HSV-2 gG2 gene in CJ2-gD2 with a codon-optimized gB2 gene under the control of the modified tetO-containing HSV-1 immediate early ICP0 promoter (US 2021/0107946; WO2019152821). CJ2-gD2/gB2 expressed gD2 as efficiently as CJ2-gD2 and expressed higher levels of gB2 than CJ2-gD2. The high effectiveness of CJ2-gD2/gB2 in protecting against wild-type HSV-2 genital infection and disease has been demonstrated in both mouse and guinea pig models of HSV-2 intravaginal infections, demonstrating that immunization can offer 100% protection against HSV-2 genital disease and is capable of eliciting a highly effective sterilizing immunity against the establishment of latent infection following intravaginal challenge with wild-type HSV-2 (US 2021/0107946). Additionally, CJ2-gD2/gB2 can serve as a very effective therapeutic vaccine against recurrent HSV-2 genital disease in guinea pigs previously infected by wild-type HSV-2 (US 2021/0107946). Importantly, purified CJ2-gD2/gB2 was as effective as un-purified CJ2-gD2/gB2 in protecting against HSV-2 genital infection and disease even at a very low dose of immunization and at challenge dose as high as 3000 LD50 (WO2019152821). Purified CJ2-gD2/gB2 was not able to establish detectable latent infection following immunization.

CJVAC, a new generation CJ2-gD2 gB2 recombinant virus that stably expresses UL9-C535C.

During the preparation and characterization of the master CJ2-gD2/gB2 viral seed, and stability evaluation of CJ2-gD2/gB2, it was learned that the HCMV promoter used to drive the expression of UL9-C535C was not stable at the HSV-2 ICP0 locus, leading to the presence of non UL9-C535C expressing and plaque-forming capable of ICP0 null mutant viruses even in low passage CJ2-gD2/gB2 stocks. With the employment of novel genome engineering, a new generation of CJ2-gD2/gB2-like HSV-2 vaccine candidates, of which CJVAC is an example, was constructed. CJVAC was highly stable in expressing UL9-C535C, completely replication-defective in normal cells, and had the same gene expressing profile as CJ2-gD2/gB2. Moreover, CJVAC encodes a novel viral replication-dependent fusogenic activity and replicates more efficiently than CJ2-gD2/gB2 in complementing tetR and ICP0 expressing cells.

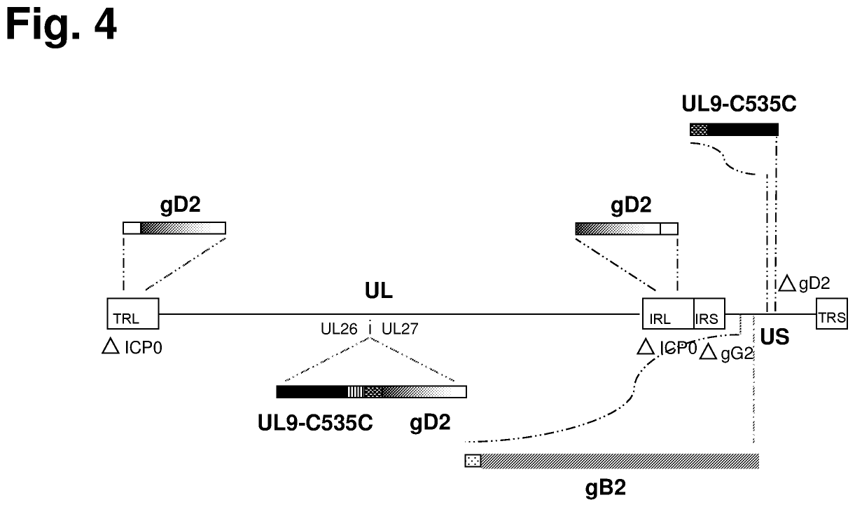

CJVAC, as described in FIG. 4, differs from CJ2-gD2/gB2 (FIG. 1) in a number of ways, including the following. The promoter used for driving UL9-C535C in CJ2-gD2/gB2 is the hCMVTO, while in CJVAC, the HSV-1 ICP27TO promoter and the HSV-1 ICP4TO are used. The locations of UL9-C535C cassette and UL9-C535C/gD2 cassette in the exemplary CJVAC virus are different from CJ2-gD2/gB2. In the exemplary CJVAC virus, the HSV-2 ICP0 gene at the HSV-2 ICP0 locus is replaced with HSV-2 gD2 gene, while in CJ2-gD2/gB2, ICP0 gene at the ICP0 locus is replaced with UL9-C535C/gD2 expressing cassette. CJ2-gD2/gB2 encodes native HSV-2 gD2 gene at the HSV-2 gD2 locus, while in CJVAC the native HSV-2 gD2 gene is deleted and replaced with UL9-C535C expressing cassette. The wild type HSV only encodes 1 copy of gD2 gene at the gD2 locus. CJVAC encodes 3 copies of gD2 gene. In CJVAC, the endogenous gD2 gene is replaced with an UL9-C535C expressing cassette, and a gD2 gene is inserted at UL26 and UL27 intergenic region, a further two copies being located at ICP0 locus.

The ICP0TO promoter used herein includes part of 5′ untranslated region of ICP0 gene as described for CJ2-gD2/gB2, and the tetO sequence is inserted 10 bp downstream of ICP0 TATA element. Thus, the tetO is located upstream of 5′ untranslated region of ICP0. SEQ ID NO: 8 is an exemplary sequence of the ICP0TO promoter. An exemplary sequence of the ICP4TO promoter is shown in SEQ ID NO:40.

Because the two UL9-C535C cassettes are inserted at different locus in the HSV-2 genome and 2 different HSV-1 promoters are used to drive the expression of UL9-C535C in the exemplary CJVAC virus, while the same HCMVTO promoter is used to drive the expression of UL9-C535C at the ICP0 locus, CJVAC is far more stable than CJ2-gD2/gB2 in expressing UL9-C535C as demonstrated in the Examples below, see, e.g., table 1.

Provided herein are compositions comprising improved viruses including the exemplary CJVAC virus and methods of using the same for immunization of subjects against infection with HSV-1 or HSV-2.

As described herein, certain of the genes in the virus are operably linked to a promoter having a TATA element. A tet operator sequence is located between 6 and 24 nucleotides 3′ to the last nucleotide in the TATA element of the promoter and 5′ to the gene. Virus may be grown in cells that express the tet repressor to block gene transcription and allow viral replication. The effectiveness of the tet repressor in blocking gene expression from the tetO sequence-containing promoter is enhanced by using a form of operator which contains two op2 elements each having the nucleotide sequence: TCCCTATCAGTGATAGAGA (SEQ ID NO: 11) linked by a sequence of, preferably, 1-3 nucleotides. When repressor is bound to this operator, very little or no transcription of the associated gene will occur. If DNA with these characteristics is present in a cell that also expresses the tetracycline repressor, transcription of the gene that can prevent viral infection and that is operably linked to the tet operator sequence (e.g., a dominant negative mutant such as UL9-C535C) will be blocked by the repressor binding to the operator and e.g. replication of the virus will occur.

During productive infection, HSV gene expression falls into three major classes based on the temporal order of expression: immediate-early (a), early (b), and late (Y), with late genes being further divided into two groups, γ1 and γ2. The expression of immediate-early genes does not require de novo viral protein synthesis and is activated by the virion-associated protein VP 16 together with cellular transcription factors when the viral DNA enters the nucleus. The protein products of the immediate-early genes are designated infected cell polypeptides ICP0, ICP4, ICP22, ICP27, and ICP47 and it is the promoters of these genes that are preferably used in directing the expression of the recombinant genes discussed herein.

ICP0 is required for efficient viral gene expression and replication at low multiplicities of infection in normal cells and efficient reactivation from latent infection (Leib et al., J Virol. 1989; 63:759-768; Cai and Schaffer, J Virol. 1989 November; 63 (11): 4579-89; Yao, et al., J. Virol. 69:6249-58 (1995)). ICP0 is needed to stimulate translation of viral mRNA in quiescent cells (Walsh and Mohr, Genes Dev. 2004 Mar. 15; 18 (6): 660-672} and plays a fundamental role in counteracting host innate antiviral response to HSV infection. In brief, it prevents an IFN-induced nuclear block to viral transcription, down regulates TLR2/TLR9-induced inflammatory cytokine response to viral infection, suppresses TNF-α mediated activation of NF-γB signaling pathway, and interferes with DNA damage response to viral infection (Lanfranca et al., Cells. 2014 June; 3 (2): 438-454). ICP4 is the major transcriptional regulatory protein of HSV, which activates the expression of viral early and late genes. ICP27 is essential for productive viral infection and is required for efficient viral DNA replication and the optimal expression of viral g genes and a subset of viral b genes. The function of ICP47 during HSV infection appears to be to down-regulate the expression of the major histocompatibility complex (MHC) class I on the surface of infected cells.

The HSV-1 UL9-C535C sequence consists of UL9 amino acids 1-10, a Thr-Met-Gly tripeptide, and amino acids 535 to 851 of UL9 (Yao et al, Hum. Gene Ther. 70:419-27 (1999)). An example of a sequence coding for UL9-C535C is provided in SEQ ID NO: 14. The other sequences described for use in recombinant viruses are all well known in the art. For example, the full length genomic sequence for HSV-1 may be found as GenBank sequence X14112. The HSV-1 ICP4 sequence may be found as GenBank number X06461; HSV-1 glycoprotein D may be found as GenBank sequence J02217; HSV-2 glycoprotein D may be found as GenBank number K01408; the HSV-1 UL 9 gene as GenBank sequence M19120; and gB2 as GenBank sequence M15118.1. All of these references are incorporated by reference herein in their entirety. Examples of gD2 and gB2 amino acid sequences are provided as SEQ ID NOs: 23 and 24 respectively.

### Inclusion of Tet Repressor and Making of Virus

Methods for making recombinant DNA molecules with genes whose expression is regulated by the tetracycline operator and repressor protein have been previously described (see U.S. Pat. Nos. 6,444,871; 6,251,640; and 5,972,650) and plasmids which contain the tetracycline-inducible transcription switch are commercially available (T-REX™, Invitrogen, CA).

An essential feature of the DNA of the present invention is the presence of genes that are operably linked to a promoter, preferably having a TATA element. A tet operator sequence is located between 6 and 24 nucleotides 3′ to the last nucleotide in the TATA element of the promoter and 5′ to the gene. Virus may be grown in cells that express the tet repressor to block gene transcription and allow viral replication. The effectiveness of the tet repressor in blocking gene expression from the tetO sequence-containing promoter is enhanced by using a form of operator which contains two op2 elements each having the nucleotide sequence: TCCCTATCAGTGATAGAGA (SEQ ID NO:11) linked by a sequence of, preferably, 1-3 nucleotides. When repressor is bound to this operator, very little or no transcription of the associated gene will occur. If DNA with these characteristics is present in a cell that also expresses the tetracycline repressor, transcription of the gene that can prevent viral infection and that is operably linked to the tet operator sequence (e.g., a dominant negative mutant such as UL9-C535C) will be blocked by the repressor binding to the operator and e.g. replication of the virus will occur.

Sequences for the HSV ICP0 and ICP4 promoters and for the genes whose regulation they endogenously control are well known in the art (McGeoch et al., J. Gen. Virol. 72:3057-3075 (1991); McGeoch et al, Nucl. Acid Res. 77:1727-1745 (1986); Perry et al., J. Gen. Virol. 67:2365-2380 (1986)) and procedures for making viral vectors containing these elements have been previously described (see US 2005/0266564). These promoters are not only very active in promoting gene expression, they are also specifically induced by VP 16, a virus-associated transactivator released when HSV-1 or HSV-2 infects a cell.

Once appropriate DNA constructs have been produced, they may be incorporated into HSV-2 virus using methods that are well known in the art (Akhrameyeva, J. Virol. 55:5036-47 (2011); Lu, et al, J. Invest. Dermatol. 729:1174-84 (2009); Yao, et al, Hum. Gene Ther. 70:1811-8 (1999)).

Viruses described herein can be replicated using complementing cells. A complementing cell expresses the gene or genes missing in the genome of a replication-defective virus (e.g., ICP0 and VP5), and are commonly used to propagate replication-defective viruses. Complementary cells are further reviewed in Dudek and Knipe. Virology 2006 January; 344 (1): 230-239. One skilled in the art will be capable of determining the appropriate complementary cell for use in replicating a given virus described herein. Preferably, the complementary cell further expresses TetR in order to repress expression from the TetO-regulated promoters. In one embodiment, the complementing cells express UL9. In another embodiment, the complementing cells express ICP0 and UL9.

The viral compositions described herein can be made, unless otherwise indicated and analyzed, using conventional techniques of molecular biology (including recombinant techniques), microbiology, cell biology, biochemistry, nucleic acid chemistry, and immunology, which are within the skill of the art. Such techniques are explained fully in the literature, such as, Current Protocols in Immunology (J. E. Coligan et al., eds., 1999, including supplements through 2016); Current Protocols in Molecular Biology (F. M. Ausubel et al., eds., 1987, including supplements through 2016); Short Protocols in Molecular Biology, F. M. Ausubel et al., eds., fifth edition 2002, including supplements through 2016; Molecular Cloning: A Laboratory Manual, third edition (Sambrook and Russel, 2001); PCR: The Polymerase Chain Reaction, (Mullis et ak, eds., 1994); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Harlow and Lane, Using Antibodies: A Laboratory Manual (Cold Spring Harbor Laboratory Press, Cold Spring Harbor, N. Y., 1999; and Beaucage et al. eds., Current Protocols in Nucleic Acid Chemistry John Wiley & Sons, Inc., New York, 2000, including supplements through 2016).

As used herein the term “herpes simplex virus” (HSV) refers to both HSV type 1 and HSV type 2 (See e.g. Fatahzadeh M I, Schwartz R A. Human herpes simplex virus infections: epidemiology, pathogenesis, symptomatology, diagnosis, and management, J Am Acad Dermatol. 2007 November; 57 (5): 737-63, ATCC holdings (Manassas, VA 20110 USA) include a number of HSV-1 and HSV-2 strains, including for example: HSV-1 HF; HSV-1 MacIntyre; HSV-1 KOS; HSV-1 GHSV-UL46; HSV-1 ATCC-2011-9; HSV-2 MS; HSV-2 G; HSV-2 186, HSV-2 ATCC-2011-2). As used herein, the term “ICP0 protein” refers to the HSV protein that is an immediate-early protein which possesses E3 ubiquitin ligase activity. ICP0 activates HSV-1 gene expression, disrupts nuclear domain (ND) 10 structures, mediates the degradation of cellular proteins, and enables evasion of the host's antiviral defenses. As used herein the term “ICP0 deficient HSV” refers to a recombinant HSV vector whose genome does not encode active ICP0 or fully functional ICP0, i.e. ICP0 with normal wild type function. Activity of ICP0 can be monitored using any of the means known to those in the art (See e.g., Smith et al, Future Virol. 2011 April; 6 (4): 421-429; Lanfranca et al., Cells 2014 3:438-454).

There are many variants of HSV ICP0 protein, e.g. some of HSV-1 ICP0, strain KOS variants are: Genebank Accession: P08393.1 GI: 124134; Accession: AFI23590.1 GI: 384597746; Accession: AFI23649.1 GI: 384597805; Accession: AFE62827.1 GI: 380776964; Accession: AFE62886.1 GI: 380777023; Accession: ADM22381.1 GI: 304318198; Accession: AL018731.1 GI: 952947655; Accession: AL018672.1 GI: 952947596; Accession: AL018655.1 GI: 952947578; Accession: AL018596.1 GI: 952947519; Accession: AKH80472.1 GI: 822581062; Accession: AKH80399.1 GI: 822580988; Accession: AKG61929.1 GI: 820021112; Accession: AKG61857.1 GI: 820021035; etc. and the like. Each strain of HSV1 or of HSV2 have multiple variants, all with functional ICP0. These variants are well known in the art and can be found in protein databases. Such variants may be used in methods of the invention. Examples of HSV-2 ICP0 variants, include but are not limited to: Accession: YP009137210.1 GI: 820945210; Accession: YP_009137151.1 GI: 820945151; Accession: AEV91397.2 GI: 556197555; Accession: AEV91338.2 GI: 556197550; Accession: ADG01890.1 GI: 295322885; Accession: ADG01889.1 GI: 295322883; Accession: ADG01888.1 GI: 295322881; Accession: ADG01887.1 GI: 295322879; Accession: ADG01885.1 GI: 295322875; Accession: ADG01886.1 GI: 295322877; etc, and the like.

As used herein, the term “gG2 protein” refers to an antigenic envelope glycoprotein that is specific for HSV-2 virus (See Gorander, S. et al, Glycoprotein G of HSV-2 as a novel vaccine antigen for immunity to genital and neurological disease). The protein has been mapped to the US segment of HSV-2 genome (See Mardsen et al., J Virol. 1984, 50 (2): 547-554 and Roizman et al. Virology, 1984, 133:242-247). gG2 protein is cleaved intracellularly into a membrane bound portion and a secreted portion. Both the membrane bound portion and the secreted portion of gG2 function as antigens (Staffan et al. J Clin. Microbiol. 2003, 41 (8): 3681-3686; Staffan et al. Clin. Vaccine Immunol. 2006, 13 (6): 633-639). The secreted portion of gG2 is also known to modify NGF-TrkA signaling to attract free nerve endings to the site of infection (Cabrera, et al. PLOS Pathog. 2015 January; 11 (1): el00457). Alternative names for HSV gG2 protein are: HSV2 gG, HSV2 gG antigen, HSV gG-2 protein, HSV gG 2, Herpes Simplex Virus 2 glycoprotein G protein, HSV-2 gG protein, HSV gG-2.

The HSV gG2 gene is also known as US4. The complete nucleotide sequence can be found at GenBank Accession: KF588470.1. In certain embodiments, gB2 is located at the gG2 (US4) locus of the HSV-2 genome thereby generating a gG2 deficient HSV-2.

As used herein the term “gG2 deficient HSV-2” or “gG2” refers to a recombinant HSV vector whose genome does not encode active or functional gG2, i.e. gG2 with wild type function, e.g. antigenic function. Serologic antigenic activity of gG2 can be monitored using any of the means known to those in the art (See e.g. Sulaiman et al, Clin Vaccine Immunol. 2009 June; 16 (6): 931-934). It should be understood that there are many variants of HSV gG2 protein, all with functional gG2. These variants are well known in the art and can be found in protein databases.

As used herein, “displaces” refers to the removal of a gene (e.g., ICP0, gG2 or gD2), or fragment thereof, from its endogenous location in the vector genome by the locatlization of an exogenous sequence (e.g., an indicated coding sequence) into such endogenous location. “Displacing” a gene can result in the depletion of the gene such that a genome no longer encodes the active or functional gene, or hinders the function of the gene.

As used herein, the term “variant” in the context of polypeptides or proteins refers to a polypeptide or protein that comprises an amino acid sequence which has been altered by the introduction of amino acid residue substitutions, deletions and/or additions. Typically, substitutions are conservative amino acid substitutions, however non-conservative substitutions can be made that do not destroy the functionality of the protein, e.g. HSV gB2 or gD2 proteins. “Conservative amino acid substitutions” refers to replacing one amino acid with another having similar structural and/or chemical properties, e.g. such as the replacement of a leucine with an isoleucine or valine, an aspartate with a glutamate, or a threonine with a serine, or glycine with another small amino acid residue. Conservative substitution tables providing functionally similar amino acids are well known in the art. As used herein, the term “non-conservative” refers to substituting an amino acid residue for a different amino acid residue that has different chemical properties. The non-conservative substitutions include, but are not limited to aspartic acid (D) being replaced with glycine (G); asparagine (N) being replaced with lysine (K); or alanine (A) being replaced with arginine (R). For purposes of embodiments of the invention non-conservative substitutions may reduce but does not destroy the proteins normal function.

As used herein the term “comprising” or “comprises” is used in reference to compositions, methods, and respective component(s) thereof, that are essential to the invention, yet open to the inclusion of unspecified elements, whether essential or not.

As used herein the terms, “consisting essentially of,” or variations such as “consists essentially of, or “consist essentially of refer to the inclusion of any recited elements, or group of elements, and the optional inclusion of other elements, of similar or different nature than the recited elements, that do not materially change the basic properties of the claimed elements. For example, a nucleotide sequence that consists essentially of a recited sequence may also include additional one or more nucleic acid additions, deletions, or substitutions that do not materially change, by a statistically significant amount, the function of the protein prior to the additions, deletions, or substitutions. For example, substitutions may correlate to the degenerative amino acid code.

The term “consisting of” refers to compositions, methods, and respective components thereof as described herein, which are exclusive of any element not recited in that description of the embodiment. For example, the nucleotide sequence has no additions, deletions or substitutions.

As used herein, the term “protein” or “polypeptide” refers to a polymer or oligomer of consecutive amino acid residues. As used herein, the term “nucleotide sequence” refers to DNA molecule sequences (e.g., cDNA or genomic DNA).

As used herein, the term “promoter” refers to regulatory control nucleic acid sequences involved in transcription of nucleotide coding sequences, which may or may not include enhancer elements. Such a promoter may be inducible or constitutive. The term “operably linked” refers to a juxtaposition wherein the components described are in a relationship permitting them to function in their intended manner. A promoter “operably linked” to a coding sequence is ligated in such a way that expression of the coding sequence is achieved.

Other than in the operating examples, or where otherwise indicated, all numbers expressing quantities of ingredients or reaction conditions used herein should be understood as modified in all instances by the term “about.” The term “about” when used in connection with percentages means±10%. In addition, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means a two-standard deviation (2SD) above or below a normal or reference level. The term refers to statistical evidence that there is a difference. The decision is often made using the p-value. If within two standard deviations than there is not a statistically significant difference.

It should be understood that this invention is not limited to the particular methodology, protocols, and reagents, and etc., described herein in the examples. The terminology used herein is for the purpose of describing particular embodiments only, and is not intended to limit the scope of the present invention, which is defined solely by the claims.

### Immunization and Treatment Methods

The viruses described herein can be used to treat or immunize subjects (e.g., mammals, e.g., humans or non-human veterinary subjects such as cats, dogs, horses, and rabbits), typically by injection as a vaccine. Other routes of administration, e.g. oral administration, could also be used. The vaccine may be used either prophylactically to prevent (reduce risk of) HSV-1 or HSV-2 infection, or therapeutically to reduce the severity of symptoms if an HSV-1 or HSV-2 infection has already occurred, e.g., as therapeutic vaccine to reduce risk, severity, or frequency of recurrent HSV-2 and HSV-1 infection in HSV-1 and HSV-2 sera positive cohorts. In order to make a vaccine, the viruses are suspended in a pharmaceutically acceptable solution such as sterile isotonic saline, water, phosphate buffered saline, 1,2-propylene glycol, polyglycols mixed with water, Ringer's solution, etc. The exact number of viruses to be administered is not crucial to the invention but should be an “effective amount,” i.e., an amount sufficient to elicit an immunological response strong enough to inhibit HSV infection. In general, it is expected that the number of viruses (PFU) initially administered will be between 1×10⁷ and 1×10⁹.

The effectiveness of a dosage, as well as the effectiveness of the overall treatment can be assessed using standard immunological methods to test for the presence of antibodies effective at attacking HSV. Immunizing injections or administrations can be repeated as many times as desired. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All references, publications and patents described herein, in the Examples and throughout the Specification, are incorporated herein by reference in their entirety. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Sequences

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Exemplary sequences include the following:
SEQ ID NO: 1: gG2 sequence: −900 to −2 bp upstream of gG2 ORF.
SEQ ID NO: 2: poly A signal of HSV-1 ICP27 sequence.
SEQ ID NO: 3: TetO-bearing modified HSV-1 ICP0 promoter plus part of 5′ untranslated region of ICP0 gene.
SEQ ID NO: 4: gG2 sequence: +1 to +900 bp downstream of gG2 ORF stop codon.
SEQ ID NO: 5: Multiple cloning sites of pCDNA3.
SEQ ID NO: 6: HSV-2 gG2 locus-specific vector sequences plus the modified tetO-bearing HSV-1 ICP0 promoter sequences in pgG2-TO. In SEQ ID NO: 06, the elements are as follows along the sequence: a) a first restriction site; b) the sequence from −900 to −2 bp upstream of the gG2 open reading frame; c) a second restriction site sequence containing one or more restriction enzyme sites; d) the poly A signal sequence of HSV-1 ICP27; e) a third restriction site sequence containing one or more restriction enzyme sites; f) the tetO-bearing modified HSV-1 ICP0 promoter plus part of 5′ untranslated region of ICP0 gene with the TATA element; g) a fourth sequence consisting of multiple restriction sites; h) the sequence from +1 to +900 bp downstream of the gG2 ORF stop codon; and h) a final restriction site.
SEQ ID NO: 7: Codon-optimized gB2 codon sequence plus Kozak consensus sequence (indicated by low and uppercase letters)
SEQ ID NO: 8: Modified HSV-1 ICP0 promoter with hCMV TATA element plus part of 5′ untranslated region of ICP0 gene (ICP0TO).
SEQ ID NO: 9: TetO-containing DNA sequence (two tandem tet operators)
SEQ ID NO: 10: Two tandem tet operator sequence.
SEQ ID NO: 11: a Tet operator op2 element;
SEQ ID NOs: 12 and 13 are TATA elements.
SEQ ID NO: 14: HSV-1 UL9-C535C coding sequence.
SEQ ID NO: 15: HSV-2 gD2 Genbank Number K01498.
SEQ ID NO: 16: HSV-1 ICP0 promoter sequence plus 5′ untranslated region of ICP0. In
SEQ ID NO: 16 the TATA element for ICP0 gene is TATAAGT.
SEQ ID NO: 17: HSV2 gD protein sequence (HSV-2, strain SD90e, GenBank: KF781518.1).
SEQ ID NO: 18: HSV2 gB protein sequence (HSV-2, strain SD90e, GenBank: KF781518.1).
SEQ ID NO: 19: HSV2 gG protein sequence (HSV-2, strain SD90e, GenBank: KF781518.1.
SEQ ID NO: 20: HSV-1 UL9 protein sequence (HSV-1, strain KOS, GenBank: JQ673480).
SEQ ID NO: 21: UL9-C535C protein sequence (MG plus UL9 amino acid 537 to 851).
SEQ ID NO: 22: HSV-1 ICP27 promoter (strain KOS).
SEQ ID NO: 23: HSV-2 ICP27 promoter (Strain HG52).
SEQ ID NO: 24: ICP4/TO promoter, codon-optimized gD2, and BGH poly A. SEQ ID NO: 24 includes the ICP4/TO promoter (nts 18-414), codon-optimized gD2 coding sequence; a part of the 3′ untranslated region of gD2; and a BGH poly A signal sequence.
SEQ ID NO:25. p2UL26.27-v.
SEQ ID NO: 26. 900 bp upstream of HSV-2 UL26 poly A signal to 110 bp downstream of UL26 poly A signal.
SEQ ID NO: 27: 121 bp downstream of HSV-2 UL27 poly A signal to 873 bp upstream of UL27 poly A signal.
SEQ ID NO: 28. A modified HSV-1 ICP27 promoter in which the ICP27 TATA element is changed to HCMV TATATAAG followed by an Age I site and part of 5′ unstranslated region of HSV-1 ICP4 and ICP27, MCS and a synthetic poly A signal sequence.
SEQ ID NO: 29: A newly designed HSV-1 tetO-bearing ICP27 promoter in which the ICP27 TATA element is changed to HCMV TATATAA and tetO sequence is flanked with Age I site followed by part of 5′ untranslated region of HSV-1 ICP4 and ICP27, MCS and a synthetic poly A signal sequence.
SEQ ID NO: 30: HSV-1 ICP27 promoter.
SEQ ID NO: 31: 5′ untranslated region of HSV-1 ICP27, −49 bp upstream of ICP27 ATG.
SEQ ID NO: 32: 5′ untranslated region of HSV-1 ICP4, −126 to −64 bp upstream of ICP4 ATG.
SEQ ID NO: 33. Multiple cloning sites:
SEQ ID NO: 34: Synthetic poly A.
SEQ ID NO: 35: p2UL2627-lacZ. In SEQ ID NO: 35, the Italic sequence represents the Hind III/Eco Rl-lacZ-containing DNA sequence inserted into p2UL2627-V.
SEQ ID NO: 36. Annotated sequence of p2ICP0-V plasmid. In SEQ ID NO: 36, nucleotide (nt) 189-nt 929 represent 5′ ICP0 flanking sequence; nt 988-nt 2388 represent 3′ ICP0 flanking sequence; and nt 930-nt 987 represents multiple cloning region. The ICP0 TATA element comprises the following sequence: TATAAGG. The ICP0 POLY A comprises the following sequence: AATAAA, which is 233 nt down stream of ICP0 stop codon TAA.
SEQ ID NO: 37: ICP4TO promoter.

### EXAMPLES

### Materials and Methods

The following materials and methods were used in the examples set forth herein.

Cells: African Green Monkey Kidney (Vero) cells and the human osteosarcoma line U2OS cells were grown and maintained in Dulbecco's Modified Eagle's Medium (DMEM; Sigma Aldrich) supplemented with 10% fetal bovine serum (FBS) in the presence of 100 U/ml penicillin G and 100 μg/ml streptomycin sulfate (GIBCO, Carlsbad, CA) (Yao, et al., J. Virol. 69:6249-58 (1995)). U2OS cells are able to complement functionally for the HSV-1 ICP0 deletion (Yao, et al., J. Virol. 69:6249-58 (1995)). U2CEP4R11 cells are tetR-expressing U2OS cells that were maintained in DMEM plus 10% FBS and hygromycin B at 50 μg/ml (Yao, et al., Hum. Gene Ther. 9:1939-50 (1998)). VOR-124 cells is a CCL-81 derived Vero cell line that stably expresses tetracycline repressor, tetR, and the HSV-1 ICP0 (See U.S. Application Ser. No. 62/515,260, Filed on Jun. 5, 2017 or PCT Application Serial No. PCT/US2018/033977, Filed on Jun. 5, 2018 entitled Vero cell lines Stably Expressing HSV ICP0 protein). V0-584 is a CCL-81 derived Vero cell line that stably expresses the HSV-1 ICP0 (See U.S. Application Ser. No. 62/515,260, Filed on Jun. 5, 2017 or PCT Application Serial No. PCT/US2018/033977, Filed on Jun. 5, 2018 entitled Vero cell lines Stably Expressing HSV ICP0 protein).

Plasmids: Plasmid p2UL2627TO is an HSV-2 UL26 and UL27 intergenic region-specific shuttle vector that contains 1) HSV-2 DNA sequence consisting of 900 bp upstream of the HSV-2 UL26 gene poly A signal to 110 bp downstream of UL26 gene poly A signal sequence, 2) DNA sequence containing a newly designed HSV-1 tetO-bearing ICP27 promoter in which the ICP27 TATA element is changed to HCMV TATATAA and tetO sequence is flanked with Age I site followed by part of 5′ unstranslated region of HSV-1 ICP4 and ICP27, MCS and a synthetic poly A signal sequence, and 3) HSV-2 DNA sequence consisting of 121 bp downstream of the HSV-2 UL27 gene poly A signal to 873 bp upstream of UL27 gene poly A signal. p2UL2627-V is a p2UL2627TO-derived plasmid, in which the tetO sequence was removed from the tetO-containing ICP27 promoter. p2UL2627-lacZ is a p2UL2627-V derived plasmid that encodes the lacZ gene under the control of the HSV-1 ICP27 promoter.

p2UL2627-C535C is a p2UL2627TO-derived plasmid that encodes the codon-optimized UL9-C535C gene under the control of the HSV-1 tetO-bearing ICP27 promoter. p2UL2627-gD2.C535C is a p2UL2627-C535C derived plasmid that encodes codon-optimized gD2 gene under the control of the tetO-containing ICP4 promoter, while the codon-optimized UL9-C535C under the control of the HSV-1 tetO-bearing ICP27 promoter.

p2ICP0-V is an HSV-2 ICP0 locus shuttle vector plasmid that consists of HSV-2 ICP0 5′-flanking sequence from nucleotides 21 bp to 762 bp upstream of ICP0 coding sequence, limited RE sites, and HSV-2 ICP0 3′-flanking sequence containing nucleotides 14 bp to 1417 bp downstream of the HSV-2 ICP0 stop codon. p2ICP0-lacZ is a p2ICP0-V derived plasmid that encodes the lacZ gene under the control of the HSV-2 ICP0 promoter. p2ICP0-gD2 is a gD2-encoding p2ICP0-V plasmid.

pgD2TO-V is an HSV-2 gD2 locus shuttle vector that consists of the C-terminal portion of codon-optimized gB2 (274 bp upstream of gB2 stop codon) plus 3′ gG2 flanking sequence extending to 125 bp downstream of US5 gene stop codon, the modified HSV-1 tetO-containing ICP4 promoter and MCS followed by DNA sequence from 54 bp upstream of gD2 stop codon to 1024 bp downstream of gD2 stop codon. pgD2TO-C535C is a p2gD2TO-V derived plasmid that encodes the codon-optimized UL9-C535C under the control of the modified tetO-bearing HSV-1 ICP4 promoter. pgD2-V is a pgD2TO-V derived plasmid in which the tetO sequence was removed from the tetO-containing ICP4 promoter by Sac I digestion followed by relegation of vector DNA. pgD2-lacZ is a LacZ gene expression plasmid derived from pgD2-V.

Viruses: Wild-type HSV-2 strain 186 was propagated and plaque assayed in Vero cells (Brans, et al., J. Invest. Dermatol. 129:2470-79 (2009); Zhang, et al., PLOS ONE, 9: e101373 (2014)). CJ2-gD2 is an HSV-2 ICP0-deletion mutant-based non-replicating dominant-negative HSV-2 recombinant virus in which both copies of the HSV-2 ICP0 gene were replaced by DNA sequences encoding the gD2 gene driven by the tetO-bearing HSV-1 major immediate-early ICP4 promoter, while the gene encoding UL9-C535C is under the control of the tetO-containing hCMV major immediate-early promoter in an opposite orientation of the inserted gD2 gene (Akhrameyeva, J. Virol. 85:5036-47 (2011); U.S. Pat. No. 8,809,047). CJ2-gD2/gB2 is a CJ2-gD2-derived HSV-2 recombinant virus in which the HSV-2 gG2 gene is replaced with the codon-optimized HSV-2 gB2 gene under the control of the tetO-containing HSV-1 ICP0 promoter (FIG. 1) (See PCT Application Serial No. PCT/US2019/016316, Filed on Feb. 1, 2019 entitled Recombinant Herpes Simplex Virus-2 expressing glycoprotein D and B antigens, and US 2021/0107946). CJ2-gD2/gB2 was propagated and plaque assayed in VOR-124 cells.

CJ2-gD2/gB2-lacZ is a CJ2-gD2/gB2-derived virus that encodes the lacZ gene under the control of the HSV-1 ICP27 promoter at the intergenic region of the HSV-2 UL26 and UL27 genes, which was generated by super-infection of Xho I/Xmn I-digested p2UL2627-lacZ and pcDNA3-tetR co-transfected U2OS cells with CJ2-gD2/gB2 at an MOI of 3 PFU/cell. The progeny viruses were plaque-purified on VOR-124 cells in the presence of 5-bromo-4-chloro-3-indolyl-b-D-galactopyranoside (X-Gal) as previously described (See PCT Application Serial No. PCT/US2019/016316, Filed on Feb. 1, 2019 entitled Recombinant Herpes Simplex Virus-2 expressing glycoprotein D and B antigens, and US 2021/0107946). Blue plaques, reflecting the insertion of the lacZ gene at the intergenic region of the UL26 and UL27 genes were isolated. CJ2-gD2/gB2-lacZ is a third round plaque-purified lacZ gene-containing CJ2-gD2/gB2 recombinant virus. The insertion of the lacZ gene in CJ2-gD2/gB2-lacZ at the intergenic region of the UL26 and UL27 genes was confirmed by PCR analyses with the primers specific for the lacZ gene and the UL27 or UL26 genes.

CJ-VAC1 is a CJ2-gD2/gB2-lacZ derived recombinant virus, in which the lacZ gene at the intergenic region of the HSV-2 UL26 and UL27 genes is replaced with DNA sequences encodes codon-optimized gD2 gene under the control of the tetO-containing HSV-1 ICP4 promoter, while the codon-optimized UL9-C535C under the control of the HSV-1 tetO-bearing ICP27 promoter. CJ-VAC1 was generated by transfection of U2CEP4R11 cells with infectious CJ2-gD2/gB2-lacZ viral DNA, Xho I/Nde I-digested p2UL2627-gD2.C535C and pCDNA3-tetR by Lipofectamine 2000-mediated transfection. Transfected cells were harvested on day 3 post-transfection followed by plaque purification on U2CEP4R-11 cells in the presence of X-Gal. White plaques reflecting the replacement of the lacZ gene at the intergenic region of the UL26 and UL27 genes with DNA encoding for gD2/UL9-C535C cassette were isolated and plaque-purified on U2CEP4R11 cells. The insertion of the codon-optimized gD2 gene and UL9-C535C gene in CJ-VAC1 at the intergenic region of the UL26 and UL27 genes was confirmed by PCR analyses with the primers specific for the codon-optimized gD2 gene, the codon-optimized UL9-C535C gene and the UL26 and UL27 genes.

Q02-gD2 is a CJ2-gD2/gB2 derived virus, in which the UL9-C535C/gD2 cassette at the ICP0 locus is replaced with a codon optimized gD2 gene, which was generated by transfection of U2CEP4R-11 cells with infectious CJ2-gD2/gB2 viral DNA and Nde I/Xho I-digested p2ICP0-gD2 and pCDNA3-tetR by Lipofectamine 2000-mediated transfection. Given that CJ2-gd2/gB2 is replication-defective in non-tetR expressing cells, the progeny viruses were selected by plaque-purification on V0-584 cells. Replacement of UL9-C535C/gD cassette with the codon-optimized gD2 gene in Q02-gD2 was confirmed by PCR analyses of Q02-gD2 viral DNA, CJ2-gD2/gB2 viral DNA and p2ICP0-gD2 plasmid DNA with 5′ primer specific for sequence upstream of ICP0 TATA box and 3′ primer specific for sequence at the end of ICP0 3′flanking sequence in p2ICP0-gD2. Q02-gD2-F is a Q02-gD2-like virus that encodes fusogenic activity.

Q02-lacZ is a CJ2-gD2/gB2 derived virus, in which the UL9-C535C/gD2 cassette at the ICP0 locus has been replaced with the lacZ gene, which was generated by transfection of U2CEP4R-11 cells with infectious CJ2-gD2/gB2 viral DNA and Asc I/Xho I-digested p2ICP0-lacZ and pCDNA3-tetR by Lipofectamine 2000-mediated transfection followed by plaque purification on V0-584 cells as previously described. Expression of the lacZ gene was confirmed by X-Gal staining. Blue plaques, reflecting the replacement of the UL9-C535C/gD cassette at the ICP0 locus with the lacZ gene were isolated followed by additional rounds of plaque-purification. Q02-lacZ-F is a Q02-lacZ virus that encodes fusogenic activity.

Q0D-D/lacZ is a Q02-gD2-derived recombinant virus, which was generated by transfection of U2OS cells with infectious Q02-gD2 viral DNA and Nar I/SnaB I-digested pgD2-lacZ followed by plaque purification in the presence of X-Gal. Blue plaques, reflecting the replacement of the gD2 gene at the gD2 locus with the lacZ gene were isolated followed by two additional rounds of plaque-purification. The replacement of the gD2 gene at the gD2 locus in Q0D-D/lacZ was confirmed by PCR analyses with the gD2 locus-specific primer and the lacZ gene specific primers. Q0D-D/lacZ-F is a fusogenic form of Q0D-D/lacZ.

CJ0D-lacZ is a Q0D-D/lacZ-derived virus that encodes the UL9-C535C/gD2 cassette at the UL26/UL27 intergenic region, the codon-optimized gD2 gene under the control of the HSV-2 ICP0 promoter at the ICP0 locus, and the lacZ gene at the gD2 locus. CJ0D-lacZ was generated by co-infection of V0R5 cells with CJ-VAC1 and Q0D-D/lacZ. Infected cells were harvested at 26 hours post-infection. The progeny viruses were plaque-purified on U2CEP4R11 cells in the presence of X-Gal. The UL9-C535C expressing blue viruses were then analyzed by infecting V0R5 cells in the presence and absence of doxycycline. The UL9-C535C-expressing viruses should be replication-defective in V0R5 cells in the presence of doxycycline and replication-competent in the absence of doxycycline. The presence of UL9-C535C/gD2 cassette at the UL26 and UL27 intergenic region in CJ0D-lacZ was confirmed by PCR analyses with the primers specific for the codon-optimized gD2 gene, the codon-optimized UL9-C535C gene and the UL26 and UL27 genes.

CJ0D-lacZ-F is a CJ0D-lacZ derived virus, which was generated by co-infection of U2CEP4R11 cells with CJ0D-lacZ and Q02-gD2-F. The progeny viruses from co-infected cells were plaque-purified on U2CEP4R11 cells in the presence of X-Gal. The blue fusogenic viruses were isolated and analyzed by their ability to replication in tetR-expressing cells in the absence and presence of doxycycline. The UL9-C535C expressing blue viruses should be replication-defective in the presence of doxycycline and replication-competent in the absence of doxycycline in U2CEP4R11 cells. The selective fusogenic blue UL9-C535C-expressing viruses were further purified by additional rounds of plaque-purification. The presence of UL9-C535C/gD2 cassette at the UL26 and UL27 intergenic region in CJ0D-lacZ-F was confirmed by PCR analyses with the primers specific for the codon-optimized gD2 gene, the codon-optimized UL9-C535C gene and the UL26 and UL27 genes.

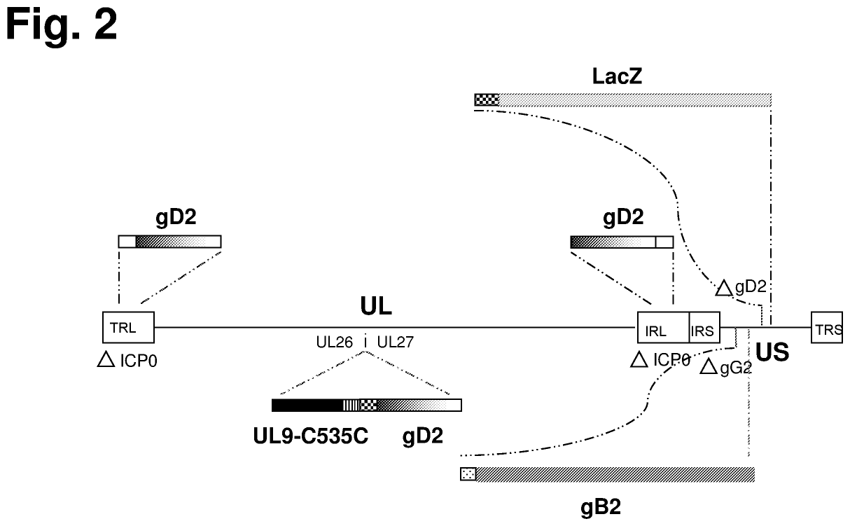

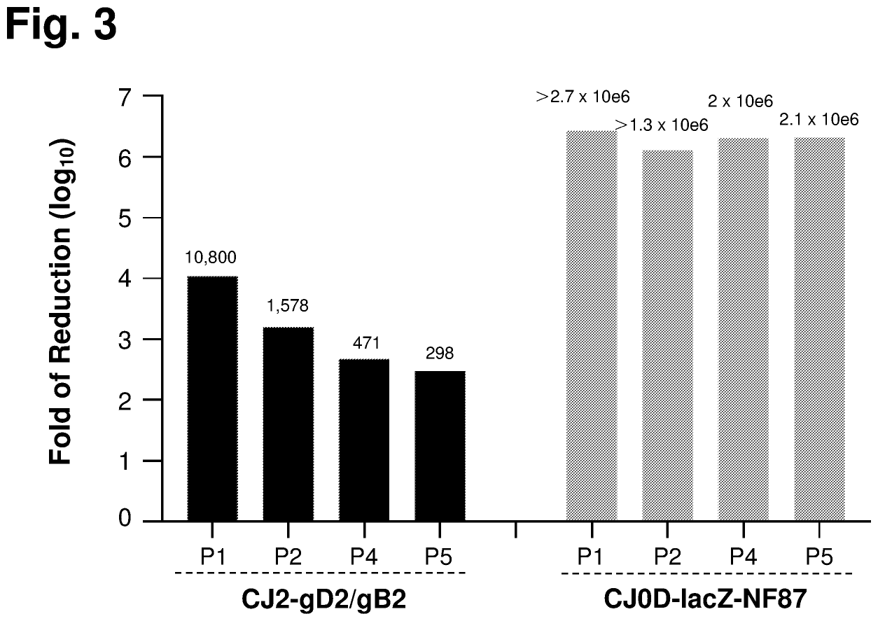

CJ0D-lacZ-NF87, is a gB2-expression gG2 minus virus that 1) encodes UL9-C535C under the control of the ICP27TO and the codon-optimized gD2 under the control of the ICP4TO at the intergenic region of UL26 and UL27 genes, 2) the second copy of the codon-optimized gD2 gene under the HSV-2 ICP0 promoter at the ICP0 locus, and 3) the lacZ gene at the gD2 locus (FIG. 2). CJ0D-lacZ-NF87 was generated by co-infection of U2CEP4R11 cells with CJ0D-lacZ-F and CJ0D-lacZ. The fusogenic progeny viruses were plaque-purified on U2CEP4R11 cells in the presence of X-Gal followed by 2 additional round of plaque-purification. The presence of UL9-C535C/gD2 cassette at the UL26 and UL27 intergenic region in CJ0D-lacZ-NF87 was confirmed by PCR analyses with the primers specific for the codon-optimized gD2 gene, the codon-optimized UL9-C535C gene and the UL26 and UL27 genes. CJ0D-lacZ-NF87 was then propagated in VOR-124 cells. The results in FIG. 3 show that CJ0D-lacZ-NF87 is significantly more stable in expressing UL9-C535C than CJ2-gD2/gB2 does as evident that titer of passage 5 CJ0D-lacZ-NF87 in the presence of doxycycline is reduced 2.1×10e6 fold compared with in the absence of doxycycline in standard plaque assay, while titer of passage 5 CJ2-gD2/gB2 in the presence of doxycycline is reduced only 298-fold compared with in the absence of doxycycline in standard plaque assay. No plaque can be detected in the presence of doxycycline when a total of 2.7×10e6 PFU of passage 1 CJ0D-lacZ-NF87 was plaque-assayed on VOR-124 monolayers in standard plaque assay.

Western blot analysis showed that CJ0D-lacZ-NF87 expresses gD2 and gB2 as efficiently as CJ2-gD2/gB2 in Vero cells (data not shown).

CJ0D-D is a Q0D-D/lacZ-F-derived virus, which encodes codon-optimized UL9-C535C under the control of the tetO-containing HSV-1 ICP4 promoter at the gD2 locus. CJ0D-D was generated by transfection of U2CEP4R11 cells with infectious Q0D-D/lacZ-F viral DNA, Nde I/SnaB I-digested pgD2TO-535C and pCDNA3-tetR by Lipofectamine 2000-mediated transfection. Progeny of transfected cells were plaque purification on U2CEP4R-11 cells in the presence of X-Gal. White plaques reflecting the replacement of the lacZ gene at the gD2 locus with DNA encoding UL9-C535C gene were isolated followed by 2 additional rounds of plaque-purification. The insertion of the UL9-C535C gene in CJ0D-D at the gD2 locus was confirmed by PCR analyses with the primers specific for the codon-optimized UL9-C535C gene and the gD2 locus. CJ0D-D-117 is a first round plaque-purified CJ0D-D virus isolated from U2CEP4R11 cells co-infected with CJ0D-lacZ-NF87 and CJ0D-D. CJ0D-D-117 exhibits significantly lower plaque-forming efficiency in U2CEP4R11 cells in the presence of doxycycline than the parental second-round plaque-purified CJ0D-D virus. Same as CJ0D-D, CJ0D-D-117 encodes codon-optimized UL9-C535C under the control of the tetO-containing HSV-1 ICP4 promoter at the gD2 locus with the intact UL26 and UL27 intergenic regions as CJ0D-D or CJ2-gD2/gB2.

### Results

Generation of CJVAC. CJVAC is a new generation gD2 and gB2-expression gG2 minus virus that encodes 1) a codon-optimized gD2 gene under the control of the HSV-2 ICP0 promoter at the ICP0 locus, 2) a UL9-C535C under the control of the ICP27TO and a codon-optimized gD2 under the control of the ICP4TO at the intergenic region of UL26 and UL27 genes, and 3) a second copy of the codon-optimized UL9-C535C gene under the control of the tetO-containing HSV-1 ICP4 promoter at the gD2 locus (FIG. 4). CJVAC was generated by co-infection of U2CEP4R11 cells with CJ0D-lacZ-NF87 and CJ0D-D-117. The resulting viral stock was diluted, filtered through a 0.22 μm filter. Viruses were then plaque-purified on U2CEP4R11 cells. Each individual plaque-purified virus was amplified in U2CEP4R11 followed by assessing its ability to replicate in U2CEP4R11 cells in either the absence or presence of doxycycline. The recombinant viruses that were completely replication-defective in U2CEP4R11 cells in the presence of doxycycline were plaque-purified by an additional round of plaque-purification. CJVAC is a second-round plaque-purified virus that is completely replication-defective in U2CEP4R11 cells in the presence of doxycycline, while replicating very efficiently in U2CEP4R11 cells in the absence of doxycycline. The presence of UL9-C535C/gD2 cassette at the UL26 and UL27 intergenic region in CJVAC was confirmed by PCR analyses with the primers specific for the codon-optimized gD2 gene, the codon-optimized UL9-C535C gene and the UL26 and UL27 genes. The presence of the UL9-C535C gene at the gD2 locus was confirmed by PCR analyses with the primers specific for the codon-optimized UL9-C535C gene and the gD2 locus. The presence of the codon-optimized gD2 gene in CJVAC at the ICP0 locus was confirmed by PCR analyses with 5′ primer specific for sequence upstream of ICP0 TATA box and primer specific for at the codon-optimized gD2 gene. The plaque-purified CJVAC was then propagated, tittered on VOR-124 cells and further analyzed as described below.

Stability of CJVAC. Our previous study with CJ2-gD2/gB2 showed that the HCMV major immediate-early promoter that drives the expression of UL9-C535C is not very stable in the context of the CJ2-gD2/gB2 genome. For example, there is an average of one plaque-forming capable ICP0- and gG2-null mutant virus in Vero cells per 1.9×10e5 PFU of passage 5 CJ2-gD2/gB2 viral stock when plaque-assayed on 6×100 mm dishes of Vero cells at a MOI of 0.083 PFU/cell (Table 1). To test the stability of CJVAC in expressing UL9-C535C, CJVAC was continuously propagated in VOR-124 cells for 10 passages. No plaque was detected in Vero cells when a total of 1.09×10e8 PFU of passage 10 CJVAC were plaque-assayed on 95×100 mm dishes of Vero cells at a MOI of about 0.085 PFU/cell (Table 1). These results indicate that CJVAC is highly stable in expressing UL9-C535C through at least passage 10.

TABLE 1
Efficiency of plaque-forming capable of
ICP0/gG2 null mutant virus in Vero cells

| | Number of PFU assayed | Number of Plaques |
|---|---|---|
| | (Based on titer in | Detected in Vero |
| Virus | V0R-124 cells) | Cells |
| CJ2-gD2/gB2 (P5) | 6.44 × 10e6 | 34 |
| CJVAC (P10) | 1.09 × 10e8 | 0 |

Vero cells were seeded at 2×10e6 cells per 100 mm dish. On day 3 post cells seeding, 1) 6×100 mm of Vero cells were infected with passage 5 CJ2-gD2-gB2 at about 1.07×10e6 PFU/dish, and 2) 47×100 mm dishes and 48 x100 mm dishes of Vero cells were infected with passage 10 CJVAC at 1.15×10e6 PFU/dish, and 1.14×10e6 PFU/dish, respectively. Infected cells were overlayered with methylcellulose and stained with neutral-red on day 4 post infection as previously described. Plaques in each individually infected dish were then counted on a light box.

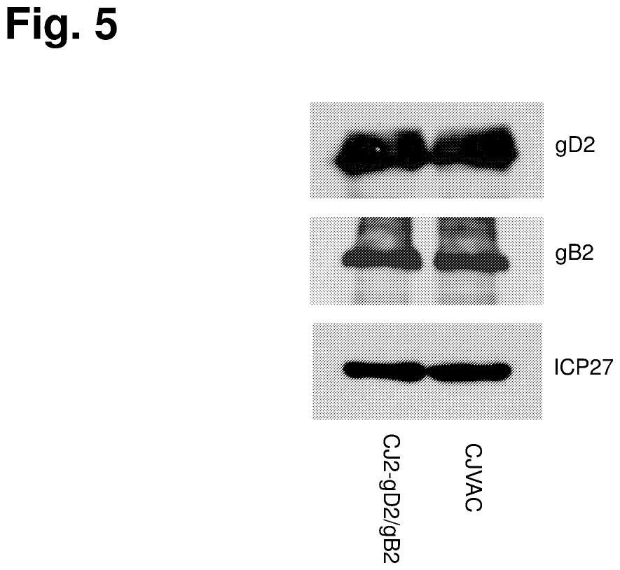

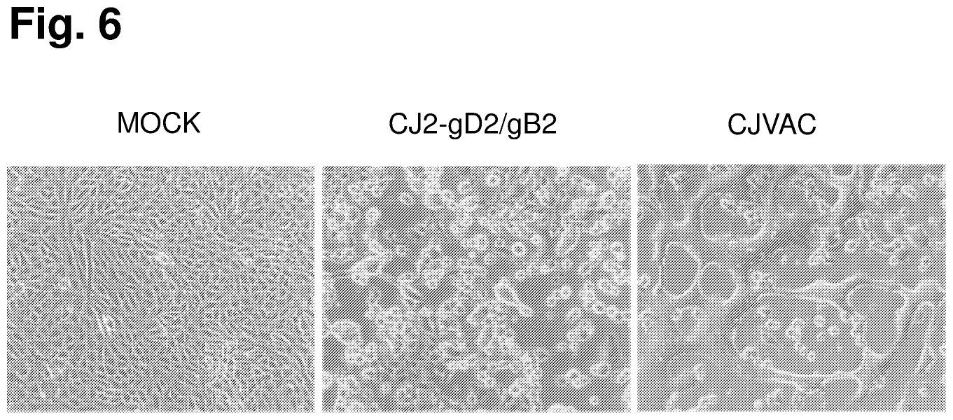

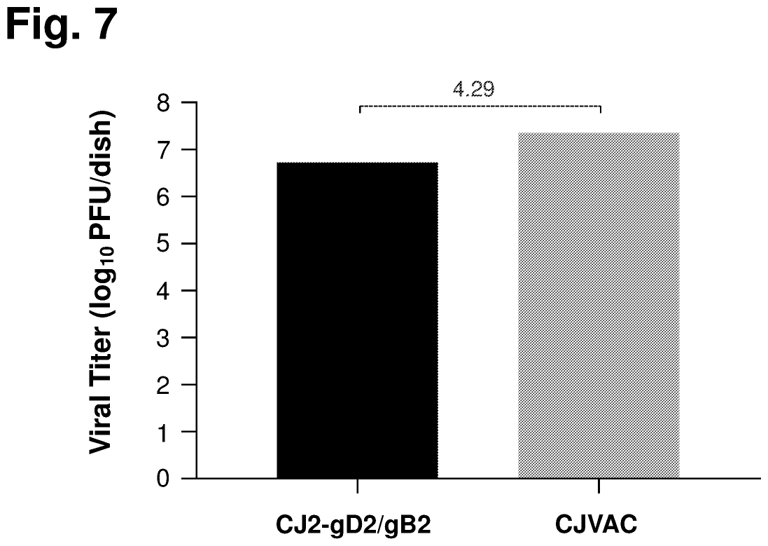

Comparisons of expression of gD2 and gB2 and replication efficiency of CJ2-gD2/gB2 and CJVAC. To examine expression of gD2 and gB2 by CJVAC, Vero cells in duplicate were infected with CJVAC and CJ2-gD2/gB2 at a MOI of 2 PFU/cells. Infected cell extracts were prepared at 20 h post-infection followed by Western blot assays with 1) anti-HSV-1/2 gB2 monoclonal antibody (10B7, sc-56987), 2) anti-HSV-1/2 gD monoclonal antibody (sc-H170), and 3) anti-ICP27 monoclonal antibody (H1142, sc-69806) as previously described. FIG. 5 showed that CJVAC expresses gD2 and gB2 as efficiently as CJ2-gD2/gB2 does. The results in FIG. 6 reveal that compared with CJ2-gD2/gB2, CJVAC encodes a fusogenic activity, leading to extensive syncytium formation in infected VOR-124 cells. The novel fusogenic activity encoded by CJVAC likely leads to more efficient viral replication in VOR-124 cells than CJ2-gD2/gB2 does at a low multiplicity of infection (FIG. 7).

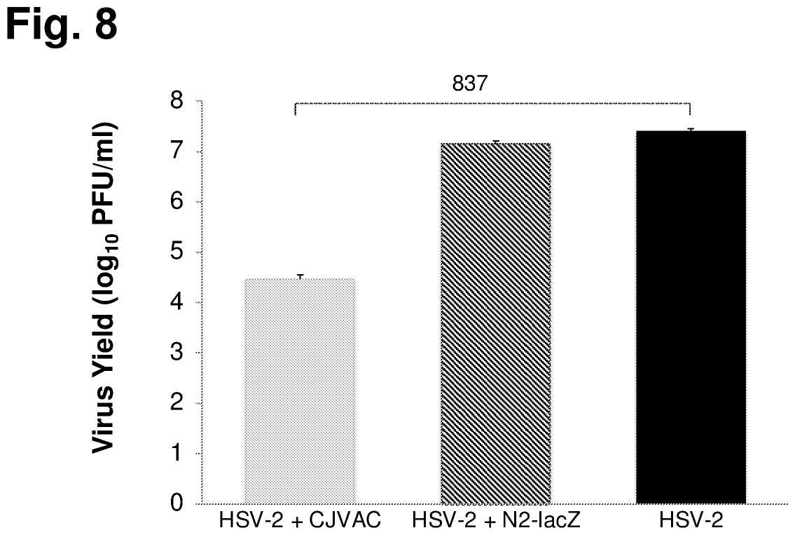

Inhibition of wild-type HSV-2 replication by CJVAC. Given the previous demonstration that overexpression of UL9-C535C by CJ2-gD2/gB2 can lead to a significantly reduction in wild-type HSV-2 viral replication in co-infected cells (See PCT Application Serial No. PCT/US2019/016316, Filed on Feb. 1, 2019 entitled Recombinant Herpes Simplex Virus-2 expressing glycoprotein D and B antigens, and US 2021/0107946), we then tested the dominant-negative effect of UL9-C535C encoded by CJVAC on the replication of wild-type HSV-2 by the co-infection assay. As shown in FIG. 8, co-infection of Vero cells with CJVAC at an MOI of 5 PFU/cell and wild-type HSV-2 at an MOI of 2 PFU/cells led to a 837-fold decrease in wild-type HSV-2 production compared with cells singly infected by wild-type HSV-2 at the same MOI. Little reduction in wild-type virus yield was detected when a similar co-infection experiment was performed with N2-lacZ. This result demonstrates that CJVAC can effectively block the replication of wild-type HSV2 in co-infected cells.

The present invention has been described in terms of example embodiments, and it should be appreciated that many equivalents, alternatives, variations, additions, and modifications, aside from those expressly stated, and apart from combining the different features of the foregoing versions in varying ways, can be made and are within the scope of the invention. While the above detailed description has shown, described, and pointed out novel features as applied to various embodiments, it will be understood that various omissions, substitutions, and changes in the form and details of the invention illustrated can be made without departing from the spirit of the disclosure. As will be recognized, certain embodiments of the disclosures described herein can be embodied within a form that does not provide all of the features and benefits set forth herein, as some features can be used or practiced separately from others. The scope of certain disclosures disclosed herein is indicated by the appended claims rather than by the foregoing description. All changes which come within the meaning and range of equivalency of the claims are to be embraced within their scope.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A replication-defective Herpes simplex virus 2 (HSV-2) recombinant virus, comprising within its genome:
a) a sequence comprising a codon optimized HSV-2 glycoprotein B2 (gB2) coding sequence operably linked to an ICP0 promoter that is operably linked to a tetO sequence, wherein the gB2 coding sequence is inserted at the gG2 locus of the HSV-2 genome;
b) a sequence comprising a codon-optimized HSV-2 glycoprotein D2 (gD2) coding sequence operably linked to an HSV-1 or HSV-2 immediate early promoter, wherein the gD2 coding sequence is inserted at the ICP0 locus;
c) a sequence comprising a first coding sequence encoding a dominant negative mutant HSV-1 or HSV-2 UL9 protein (dnUL9) operably linked to a ICP27 promoter that is operably linked to a tetO sequence and a codon-optimized gD2 sequence operably linked to an ICP4 promoter that is operably linked to a tetO sequence, wherein the dnUL9/gD2 sequence is inserted into the intergenic region of the HSV-2 UL26 and UL27 genes; and
d) a sequence comprising a second dnUL9 coding sequence operably linked to an ICP4 promoter that is operably linked to a tetO sequence wherein the second UL9-C535C coding sequence is inserted at the gD2 locus,
wherein the genome of the virus does not comprise a sequence encoding a functional endogenous ICP0 protein, a functional endogenous HSV-2 gG2 protein.

2. The recombinant virus of claim 1, wherein the genome of the virus does not comprise an endogenous sequence encoding a functional HSV-2 gD2 protein.

3. The recombinant virus of claim 1, wherein any one or more of the gB2, gD2, UL9-C535C/gD2, and UL9-C535C coding sequences are codon optimized.

4. The recombinant virus of claim 1, wherein the ICP0 promoter that is operably linked to a tetO sequence in part a) comprises SEQ ID NO:8.

5. The recombinant virus of claim 1, wherein the HSV-1 or HSV-2 immediate early promoter in b) is selected from the group consisting of an ICP0 promoter, an ICP27 promoter, and an ICP4 promoter.

6. The recombinant virus of claim 5, wherein the HSV-1 or HSV-2 immediate early promoter in b) is a HSV-1 or HSV-2 ICP0 promoter.

7. The recombinant virus of claim 1, wherein the dominant negative mutant HSV-1 or HSV-2 UL9 protein is UL9-C535C.

8. A vaccine comprising the recombinant virus of claim 1 in unit dose form.

9. A method of immunizing a subject against HSV-1 or HSV-2 infection or treating an HSV-1 or HSV-2 infection in a subject, the method comprising administering to the subject the vaccine of claim 8.

10. The method of claim 9, wherein the subject is seropositive for HSV-1.

11. The method of claim 9, wherein the subject is seropositive for HSV-2.

12. The method of claim 9, wherein the subject is seronegative for HSV-1 and HSV-2.

13. A method for producing the virus of claim 1, the method comprising; a) infecting complementing cells with the virus, wherein the complementing cells express a functional gene product or products that are needed for replication of the virus and for which sequences encoding such are lacking from the virus genome; b) culturing the complementing cells such that the virus replicates; and c) harvesting the replicated virus from the complementing cells.

14. The method of claim 13, wherein the complementary cells further express TetR.

15. The method of claim 13, wherein the complementary cells express ICP0 functional gene product.

16.-20. (canceled)
